(19) United States
(12) Patent Application Publication
(10) Pub. No.: US 2026/0335469 A1
(43) Pub. Date: Oct. 29, 2026

(54) MACROCYCLIC GADOLINIUM-BASED CONTRAST AGENTS HAVING PROLONGED CHELATE INTEGRITY AND METHODS FOR PREPARING AND ADMINISTERING THE SAME

(71) Applicant: INVENTURE, LLC, Southbury, CT (US)

(72) Inventors: Richard DESLAURIERS, Woodbury, CT (US); Paul Lombardo, Stamford, CT (US); Arno Georg Steinig, Carmel, NY (US)

(73) Assignee: INVENTURE, LLC, Southbury, CT (US)

(21) Appl. No.: 19/566,513

(22) Filed: Mar. 13, 2026

Related U.S. Application Data
(60) Provisional application No. 63/771,994, filed on Mar. 14, 2025.
(60) Provisional application No. 63/911,971, filed on Nov. 5, 2025.

(51) Int. Cl. A61K 49/10; A61K 9/00; A61K 9/08

(52) U.S. Cl. CPC A61K 49/106; A61K 9/0019; A61K 9/08

(57) ABSTRACT

A method includes contacting a solution comprising a macrocyclic chelated gadolinium-based contrast agent (GBCA) with an agent configured to induce dechelation of Gd ions from partially coordinated complexes of the GBCA and bind dissociated Gd ions; separating the agent from the solution; and administering the solution to a patient while the solution comprises a decreased proportion of partially coordinated complexes of the GBCA relative to the solution prior to the contacting.

### CROSS-REFERENCE TO RELATED APPLICATIONS

This application claims the benefit of U.S. Provisional Patent Application No. 63/911,971, filed Nov. 5, 2025, and U.S. Provisional Patent Application No. 63/771,994, filed Mar. 14, 2025, the entire contents of each of the foregoing applications being hereby incorporated by reference herein.

### BACKGROUND OF THE DISCLOSURE

### 1. Field of the Disclosure

The present disclosure is directed to pharmaceutical formulations of contrast agents used in magnetic resonance imaging (MRI). More particularly, the present disclosure is directed to macrocyclic gadolinium-based contrast agents (GBCAs) having prolonged chelate integrity and methods of making and administering the same.

### 2. Description of Related Art

MRI relies on detecting a signal from water hydrogen nuclei under magnetic resonance conditions. Gd ion is a good MRI contrast agent because of its unique atomic configuration. This property improves image contrast by enhancing the magnetic resonance signal from surrounding water hydrogen nuclei.

GBCAs are intravenous pharmaceutical formulations used in MRIs to improve diagnostic quality by improving the visibility of structures and fluids in the body. GBCAs enable essential clinical diagnosis for numerous diseases that might otherwise go undetected.

Examples of macrocyclic GBCAs include gadoterate, gadobutrol, gadoteridol, gadopiclenol, and gadoquatrane. Each has a macrocyclic chelator or ligand complexed with gadolinium. Gadoterate, gadobutrol, and gadoteridol have ligands with 8-coordinate sites. Gadopiclenol and gadoquatrane have ligands with 7-coordinate sites.

Gadoterate, Gd-DOTA, has gadolinium ion complexed to the macrocyclic ligand 1,4,7,10-tetraazacyclododecane-1,4,7,10-tetraacetic acid (DOTA). The ligand has four nitrogen donors from a macrocyclic ring and four oxygen donors from carboxylate groups. Gadoterate is ionic. Meglumine is used as a counterion.

Gadobutrol, Gd-DO3A-butrol, has gadolinium ion complexed to the macrocyclic ligand 1,4,7,10-tetraazacyclododecane-1,4,7-triacetic acid butrolamide (DO3A-butrol). The ligand has four nitrogen donors from a macrocyclic ring, three oxygen donors from carboxylate groups, and one oxygen donor from butrolamide.

Gadoteridol, Gd-HP-DO3A, has gadolinium ion complexed to the macrocyclic ligand 1,4,7,10-tetraazacyclododecane-1,4,7-trisacetic acid, 10-hydroxypropyl (HP-DO3A). The ligand has four nitrogen donors from a macrocyclic ring, three oxygen donors from carboxylate groups, and one oxygen donor from a hydroxypropyl group.

Gadopiclenol, Gd-PCTA, has gadolinium ion complexed to the macrocyclic ligand 1,4,7,10-tetraazacyclododecane-1,4,7-trisacetic acid, 10-picolinamide (PCTA). The ligand has four nitrogen donors from a macrocyclic ring, two oxygen donors from carboxylate groups, and one nitrogen donor from picolinamide.

Gadoquatrane, Gd-GlyMe-DOTA, has four gadolinium ions complexed to four macrocyclic ligands. Each ligand has seven coordination sites.

In aqueous solution, the gadolinium ion exists as a hydrated ion with 8 to 9 coordinated water molecules in its hydration sphere.

During the synthesis of macrocyclic GBCAs, the macrocyclic chelators or ligands form “cages” around the gadolinium ion.

During a typical Gd-DOTA synthesis, for example, there is a stepwise binding of the gadolinium ion to the DOTA so that, in turn, the DOTA forms a cage around the gadolinium. Fully coordinated complexes in which the gadolinium ion is bound to all eight coordination sites of the macrocyclic chelator result from the synthesis. In a fully coordinated complex, there is a single water molecule in the inner coordination sphere. These are maximally coordinated cages with the highest coordination saturation. However, partially coordinated complexes in which the Gd ion is bound to less than all coordination sites of the macrocyclic chelator also occur. In a partially coordinated complex, there is more than one water molecule in the inner coordination sphere. These are less than maximally coordinated cages.

Further, “reaction by-products” can form due to impurities in the ligand, other metal ions present in the lanthanide raw material, or solvents used during extraction or crystallization steps. Still further, some unreacted gadolinium can remain.

When macrocyclic GBCAs are injected into a patient, the human body accelerates dechelation of the partially coordinated complexes. As a result, gadolinium can dissociate from the ligand and exist in free form.

Ferritin and other proteins found in cells and blood are excellent chelators that will readily bind to free gadolinium ions in the human body. In addition, metal cations such as Zinc (Zn²⁺), Copper (Cu²⁺), Calcium (Ca²⁺), or Iron (Fe³⁺) in the blood can displace the gadolinium from, for example, Gd-DOTA and form chelated complexes of Zn-DOTA, Cu-DOTA, Ca-DOTA, and Fe-DOTA in a process known as transmetallation. As a result, gadolinium can dissociate from the ligand, bind with ferritin or other chelators in the blood, and stay in the body as they are not excreted in the same way as the fully coordinated cages.

Gadolinium ion is highly toxic when released in its free form in the body. Gadolinium is a heavy metal that is toxic to humans. This toxicity is due in part to its ability to form nanoparticles that can enter cells and interfere with cellular function. The size and charge of gadolinium ions can mimic calcium ions. This allows gadolinium ions to substitute for calcium ions in cellular reactions, thereby disrupting important biological processes. Trace amounts of gadolinium ions can accumulate in the brain, bones, and skin. In cases where repeated administration of the GBCA is needed in patients who require regular scans, health risks increase with each administration. There is also an increased health risk when excretion is delayed, which can occur in patients who have impaired renal function.

Prior approaches have sought to mitigate the release of free gadolinium ions in the body.

As an example, one approach has been to add an excess of a chelating compound to a lanthanide-based contrast agent to reduce the amount of free gadolinium ions in the drug product before injection. However, this is not an ideal solution. First, the chelating compound itself is toxic, and thus adding excess chelate is not desirable. Second, excess chelate does not correlate with a reduced gadolinium ion release in the body.

In the vial, i.e., before administration to a patient, macrocyclic GBCAs with both fully and partially coordinated complexes appear to be stable because even the partially coordinated complexes have high thermodynamic stability constants when unchallenged by a competitive chelator such as those in the body. The measurable free gadolinium concentration in the vial can be very low. While there can be a perceived safety enhancement with respect to drug product testing and characterization, such safety enhancement is not, in fact, realized in practice when the chelates are injected into the human body.

Accordingly, it has been determined by the present disclosure that there is a continuing need for macrocyclic GCBAs having prolonged chelate integrity and methods of making and administering the same that overcome, alleviate, and/or mitigate one or more of the aforementioned and/or other deleterious effects that were the target of prior attempts.

### SUMMARY OF THE DISCLOSURE

The present disclosure has found that even after advanced processing and purification, strong cages of macrocyclic chelated GBCAs will degrade to a base equilibrium, generally understood to be from 97% to less than 99% fully coordinated complexes, colloquially “strong cages”, and greater than 1% to 3% partially coordinated complexes, colloquially “weak cages”. This degradation occurs, in part, due to the interaction between the gadolinium complexes and water or water-like molecules over time.

The present disclosure has found that strong cages are more dechelation-resistant than weak cages.

The present disclosure has found that, given all things being equal, such as UV radiation, pH, temperature, and the presence of other ions like Mg and Ca, degradation occurs due to the interaction between the macrocyclic chelated GBCA complexes and water or water-like molecules over time.

Without wishing to be bound by a single theory, the integrity of macrocyclic chelated GBCA complexes is strongly influenced by their interaction with water and water-like molecules. Macrocyclic chelated GBCA complexes typically contain one inner sphere water molecule directly coordinated to the gadolinium ion, which undergoes rapid exchange with surrounding bulk water. This process, quantified by the water residency time (τM) or the water exchange rate (k_ex), plays a crucial role in the integrity and relaxivity of the complex. The hydration state (q), which represents the number of water molecules directly coordinated to the gadolinium ion, also affects integrity, with most clinical GBCAs having a hydration state of 1. In addition to inner sphere interactions, gadolinium complexes engage with outer-sphere water molecules, further influencing their relaxivity and long-term integrity. Over time, these water interactions can contribute to the dissociation of the gadolinium-ligand complex, leading to the release of free gadolinium ions. The rate and extent of dissociation are influenced by thermodynamic and kinetic factors, as well as environmental conditions such as pH and temperature, which can impact the complex's integrity in physiological and storage environments.

The dissociation reaction of macrocyclic chelated GBCA complex in solution from fully coordinated complex to partially coordinated complex to free ion and ligand can be conceptually represented by equation 1 below:

Fully coord Gd-L [symbol] partially coord Gd-L [symbol] Gd³⁺+L (1)

In the equations above, Gd is gadolinium. L is a ligand, for example, DOTA, DO3A-butrol, HP-DO3A, PCTA, and GlyMe-DOTA.

Without wishing to be bound by theory, it is believed that the conditional stability constants of the partially coordinated complexes are much lower than for the fully coordinated complexes. When free gadolinium ions are chelated in the blood by proteins, including ferritin, the equilibrium is disrupted, and the system will respond by shifting to produce more free ions to restore the original ratio and will drive the dissociation from the partially coordinated complexes to restore the equilibrium. In turn, the fully coordinated complexes will convert to partially coordinated complexes to restore their equilibrium concentrations. The present disclosure has found that over time, the system will adjust until the concentration of partially coordinated complexes reaches the 1 to 3% level again, whereby some gadolinium from the fully coordinated complex will dissociate from being fully coordinated to partially coordinated to compensate.

These interactions contribute to the degradation of fully coordinated complexes having strong cages into partially coordinated complexes having weak cages, which are more prone to dissociation and the release of free gadolinium. This degradation presents a significant challenge for the long-term storage and efficacy of GBCAs. By addressing these integrity concerns, the present disclosure improves the durability of gadolinium complexes, thereby enhancing their safety and reducing the risk of gadolinium deposition in biological tissues.

A macrocyclic chelated GBCA complex containing partially coordinated complexes at levels as low as 0.05%, meaning that 99.95% of the complexes are fully coordinated complexes, can be described as a high-purity substance with “4N” purity. The term “4N” refers to the number of nines in the purity percentage, with 99.95% corresponding to four nines, i.e., 4N.

However, the present disclosure has found that a product with 4N purity exists only for a limited period, typically 12 to 48 hours, due to the tendency to equilibrate with partially coordinated complexes. When water is present, the equilibrium concentration typically approaches a steady state at 97 to 99% fully coordinated complexes and 1 to 3% partially coordinated complexes. Without wishing to be bound by a single theory, this shift toward equilibrium is driven by several factors. First, a dynamic equilibrium exists even in highly pure solutions, wherein complexes continuously form and break down at the molecular level. Second, despite the high thermodynamic and kinetic integrity of, for example, gadoterate meglumine, with a log K(therm) value of 25.6 and a log K(cond) value of 19.3, these values account for relative concentrations of raw materials (such as Gd ion) and the final product but do not consider or distinguish fully and partially coordinated complexes. These are at pH 7.4 and without the presence of competitive chelators. Lastly, the presence of a high concentration of water molecules in the solution increases the likelihood of interactions with fully coordinated complexes in which the gadolinium ion is bound to all eight coordination sites of the macrocyclic chelator and contains only one water molecule in its inner sphere.

This temporary state highlights the inherent challenges in maintaining chelate integrity in GBCAs, even with advanced purification techniques. It underscores the difficulties in preserving 4N purity at the time of use, as equilibrium shifts inevitably introduce partially coordinated complexes into the solution. It is these partially coordinated complexes that can quickly release their free complexed gadolinium in the first few hours after injection, which is believed to be a key mechanism leading to gadolinium deposition in various tissues, including the brain, bones, and skin. The present disclosure has found that by minimizing or delaying the formation of partially coordinated complexes, and/or removing partially coordinated complexes at the time of use in the clinic, the risk of gadolinium deposition can be reduced, thereby enhancing the safety profile of macrocyclic GBCAs.

Without wishing to be bound by theory, it is believed that partially coordinated complexes break down in vivo before being excreted, while fully coordinated complexes do not break down before excretion. Thus, preparing GBCA solutions containing all or mostly strong cages is preferred because strong cages are less likely to release toxic free gadolinium ions before excretion from the body.

It has been found that the direct manufacture of gadolinium chelate solutions without weak cages is effectively impossible due to the equilibrium and reaction chemistry effects. This disclosure has found a rapid and efficient method for removing weak cages and free gadolinium ions from aqueous solution, leaving behind the desired strong cages. This solution can be administered before equilibrium is reestablished. Contrast agent solutions with higher initial strong cage concentrations prolong the time needed to release free gadolinium ions, offering greater safety during use as a contrast agent.

The pharmaceutical formulations of the present disclosure are intended to be free or substantially free of partially coordinated complexes, thereby ensuring the presence of fully coordinated complexes. In this context, substantially free means In this context, substantially free means less than 1%, less than 0.9%, less than 0.8%, less than 0.7%, less than 0.6%, less than 0.5%, less than 0.4%, less than 0.3%, less than 0.2%, less than 0.1%, less than 0.075%, less than 0.05%, less than 0.025%, less than 0.01%, less than 0.0075%, less than 0.005%, less than 0.0025%, less than 0.001%, less than 0.0001%, or less than 0.00001% partially coordinated complexes. Conversely, there are at least 99%, at least 99.1%, at least 99.2%, at least 99.3%, at least 99.4%, at least 99.5%, at least 99.6%, at least 99.7%, at least 99.8%, at least 99.9%, at least 99.925%, at least 99.95%, at least 99.975%, at least 99.99%, at least 99.9925%, at least 99.995%, at least 99.9975%, at least 99.999%, at least 99.9999%, or at least 99.99999% fully coordinated complexes.

The present disclosure further provides methods for maintaining superior kinetic and conditional thermodynamic integrity over a commercially viable shelf life, improving upon prior formulations. These macrocyclic chelated GBCA complexes have been found to be particularly well-suited for use in MRI contrast agents due to their prolonged chelate integrity and reduced susceptibility to degradation, ultimately being a safer product.

The present disclosure unexpectedly found that certain methods discussed herein preserve the chelate integrity of the macrocyclic chelated GBCA complexes.

Chelate integrity can be enhanced by exposure of the GBCA to a selective exchange medium, which is referred to herein as exchange media treatment.

A selective exchange medium is any resin, membrane, monolith, particle, bead, or other solid phase whose functional groups have a preferential affinity for trivalent cations, such as Gd³⁺, and preferably more affinity relative to tetravalent, divalent, or monovalent cations. The selective exchange mediums of the present disclosure are configured to induce dechelation of Gd ions from partially coordinated GBCA complexes and to capture the free Gd ions. The selective exchange mediums can also be configured to isolate and remove partially coordinated complexes of the GBCA in the solution, to isolate and remove free macrocyclic chelate in the solution, or both. Preferably, the selective exchange medium is trivalent-cation selective.

The disclosure has unexpectedly found that exchange media treatment effectively eliminates weak cages. According to conventional understanding, dechelated gadolinium ions should bind, for example, to a resin, while the chelating agent, DOTA, would remain in solution. However, the present disclosure has found that the concentration of free DOTA remains unchanged. Rather, in some example embodiments, the exchange media of the present disclosure remove intact, but weak cages rather than just isolated metal ions, chelating agents, or both, all without affecting strong cages.

For example, at sub-freezing temperatures, the equilibrium chemistry of the macrocyclic chelated GBCA complexes slows, thereby reducing the rate of structural degradation from fully coordinated complexes to partially coordinated complexes.

As another example, by removing the bulk of water from the macrocyclic chelated GBCA complexes, equilibrium-based degradation of fully coordinated complexes into the weaker, partially coordinated complexes can be slowed.

As used herein, a “dry” macrocyclic chelated GBCA refers to a solid or substantially solid composition in which the gadolinium chelate retains its coordinated inner-sphere water molecule (q=1), while free or uncoordinated water is limited to less than about 5 wt %, less than about 4 wt %, less than about 3 wt %, less than about 2 wt %, less than about 1 wt %, or less than about 0.5 wt %. In certain embodiments, the GBCA is dry gadoterate meglumine having one inner-sphere water molecule and less than about 5 wt %, less than about 4 wt %, less than about 3 wt %, less than about 2 wt %, less than about 1 wt %, or less than about 0.5 wt % free water.

Without wishing to be bound by theory, reducing the amount of free water in a macrocyclic gadolinium contrast agent can slow degradation by limiting hydrolysis, reducing solvent-mediated reactions, and physically maintaining the complex's structural integrity.

As another example, exposing macrocyclic chelated GBCA complexes to selective exchange media during storage enables continuous removal of degradation products arising from partially coordinated complexes, thereby further prolonging the chelate integrity of the formulation.

Herein, these, together with the methods discussed below, individually or collectively contribute to achieving MRI contrast agents with improved shelf life, chelate integrity, and safety.

The present disclosure provides a dry composition suitable for reconstitution into a pharmaceutically acceptable injectable solution. The composition has a macrocyclic chelated gadolinium-based contrast agent (GBCA). The GBCA is dry and characterized in that, based on total GBCA complexes present, at least 99% mol are fully coordinated complexes in which Gd ion is bound to all coordination sites of the macrocyclic chelator, and not more than 1% mol are partially coordinated complexes in which Gd ion is bound to fewer than all coordination sites of the macrocyclic chelator.

In some embodiments according to the disclosure, either alone or together with any one or more of the aforementioned and/or after-mentioned embodiments, the GBCA has less than 5 wt % free water.

In some embodiments according to the disclosure, either alone or together with any one or more of the aforementioned and/or after-mentioned embodiments, the GBCA has less than 3 wt % free water.

In some embodiments according to the disclosure, either alone or together with any one or more of the aforementioned and/or after-mentioned embodiments, the GBCA retains a single coordinated inner-sphere water molecule characteristic of a fully coordinated GBCA complex.

In some embodiments according to the disclosure, either alone or together with any one or more of the aforementioned and/or after-mentioned embodiments, the GBCA is selected from the group consisting of: gadoterate, gadobutrol, gadoteridol, gadopiclenol, and gadoquatrane.

The present disclosure provides a dry composition suitable for reconstitution into a pharmaceutically acceptable injectable solution. The composition has a macrocyclic chelated gadolinium-based contrast agent (GBCA). The GBCA is dry and characterized in that, based on total GBCA complexes present, at least 99% mol are fully coordinated complexes in which Gd ion is bound to all coordination sites of the macrocyclic chelator, and not more than 1% mol are partially coordinated complexes in which Gd ion is bound to fewer than all coordination sites of the macrocyclic chelator. The composition further includes an agent configured to, upon hydration of the GBCA, induce dechelation of Gd ions from partially coordinated complexes of the macrocyclic chelated GBCA and bind dissociated Gd ions. The GBCA and the agent are both in a dry state, and the agent remains substantially unreactive in the absence of free water and becomes effective upon hydration.

In some embodiments according to the disclosure, either alone or together with any one or more of the aforementioned and/or after-mentioned embodiments, the agent is a trivalent-cation selective resin.

In some embodiments according to the disclosure, either alone or together with any one or more of the aforementioned and/or after-mentioned embodiments, the agent reduces a proportion of partially coordinated complexes of the GBCA.

A drug product includes a syringe having a barrel defining at least a first chamber and a second chamber separated by a barrier. The drug product also includes a dry macrocyclic chelated gadolinium-based contrast agent (GBCA) and a dry selective exchange medium disposed in the first chamber. The GBCA is characterized in that, based on total GBCA complexes present, at least 99% mol are fully coordinated complexes in which Gd ion is bound to all coordination sites of the macrocyclic chelator, and not more than 1% mol are partially coordinated complexes in which Gd ion is bound to fewer than all coordination sites of the macrocyclic chelator. A pharmaceutically acceptable aqueous carrier disposed in the second chamber. The barrier is configured to permit fluid communication between the chambers so that the aqueous carrier is capable of contacting the dry GBCA and the dry selective exchange medium to form a reconstituted GBCA solution within the syringe.

In some embodiments according to the disclosure, either alone or together with any one or more of the aforementioned and/or after-mentioned embodiments the barrier comprises a frangible seal or a bypass channel.

In some embodiments according to the disclosure, either alone or together with any one or more of the aforementioned and/or after-mentioned embodiments the selective exchange medium comprises a cation exchange resin, an ion exchange membrane, a monolith, ion exchange bead, ion exchange particle, a porous matrix, a functionalized polymer, or a mixed-bed ion exchanger.

In some embodiments according to the disclosure, either alone or together with any one or more of the aforementioned and/or after-mentioned embodiments, the drug product has a screen, frit, or filter configured to retain the selective exchange medium within the syringe while permitting the reconstituted GBCA solution to exit through an outlet of the syringe.

In some embodiments according to the disclosure, either alone or together with any one or more of the aforementioned and/or after-mentioned embodiments the pharmaceutically acceptable aqueous carrier comprises water for injection, saline, dextrose in water, or sodium lactate solution.

In some embodiments according to the disclosure, either alone or together with any one or more of the aforementioned and/or after-mentioned embodiments the GBCA is selected from the group consisting of: gadoterate, gadobutrol, gadoteridol, gadopiclenol, and gadoquatrane.

In some embodiments according to the disclosure, either alone or together with any one or more of the aforementioned and/or after-mentioned embodiments the selective exchange medium is configured to, upon hydration of the GBCA, induce dechelation of Gd ions from partially coordinated complexes of the macrocyclic chelated GBCA and bind dissociated Gd ions.

In some embodiments according to the disclosure, either alone or together with any one or more of the aforementioned and/or after-mentioned embodiments the barrel further defines a third chamber and comprises a further barrier, the barrier is positioned between the first chamber and the second chamber, the further barrier is positioned between the second chamber and the third chamber, and the pharmaceutically acceptable aqueous carrier, the selective exchange medium, and the dry GBCA are separately disposed in the first, second, and third chambers, respectively.

In some embodiments according to the disclosure, either alone or together with any one or more of the aforementioned and/or after-mentioned embodiments, the syringe comprises a plunger and actuation of the plunger causes the aqueous carrier to flow from the second chamber into the first chamber to reconstitute the GBCA and contact, thereby activating the selective exchange medium.

A method according to the present disclosure includes contacting a solution comprising a macrocyclic chelated gadolinium-based contrast agent (GBCA) with an agent configured to induce dechelation of Gd ions from partially coordinated complexes of the GBCA and bind dissociated Gd ions, separating the agent from the solution, and administering the solution to a patient while the solution comprises a decreased proportion of partially coordinated complexes of the GBCA relative to the solution prior to the contacting.

In some embodiments according to the disclosure, either alone or together with any one or more of the aforementioned and/or after-mentioned embodiments, the agent is further configured to isolate and remove partially coordinated complexes of the GBCA in the solution.

In some embodiments according to the disclosure, either alone or together with any one or more of the aforementioned and/or after-mentioned embodiments, the agent is further configured to isolate and remove free macrocyclic chelate in the solution.

In some embodiments according to the disclosure, either alone or together with any one or more of the aforementioned and/or after-mentioned embodiments, the agent is further configured to isolate and remove partially coordinated complexes of the GBCA in the solution and free macrocyclic chelate in the solution.

In some embodiments according to the disclosure, either alone or together with any one or more of the aforementioned and/or after-mentioned embodiments, the administering is performed within 24 hours of the contacting.

In some embodiments according to the disclosure, either alone or together with any one or more of the aforementioned and/or after-mentioned embodiments, the administering is performed while at least 99% mol are fully coordinated complexes in which Gd ion is bound to all coordination sites of a macrocyclic chelator of the GBCA and at most 1% mol are partially coordinated complexes in which Gd ion is bound to fewer than all coordination sites of the macrocyclic chelator.

In some embodiments according to the disclosure, either alone or together with any one or more of the aforementioned and/or after-mentioned embodiments, where at least 99% mol of total GBCA complexes are fully coordinated complexes in which Gd ion is bound to all coordination sites of the macrocyclic chelator, and not more than 1% mol are partially coordinated complexes in which Gd ion is bound to fewer than all coordination sites of the macrocyclic chelator, the proportion of partially coordinated complexes is not more than 1% mol, less than 0.9% mol, less than 0.8% mol, less than 0.7% mol, less than 0.6% mol, less than 0.5% mol, less than 0.4% mol, less than 0.3% mol, less than 0.2% mol, less than 0.1% mol, less than 0.075% mol, less than 0.05% mol, less than 0.025% mol, less than 0.01% mol, less than 0.0075% mol, less than 0.005% mol, less than 0.0025% mol, less than 0.001% mol, less than 0.0001% mol, or less than 0.00001% mol. In these embodiments, the proportion of fully coordinated complexes is at least 99% mol, at least 99.1% mol, at least 99.2% mol, at least 99.3% mol, at least 99.4% mol, at least 99.5% mol, at least 99.6% mol, at least 99.7% mol, at least 99.8% mol, at least 99.9% mol, at least 99.925% mol, at least 99.95% mol, at least 99.975% mol, at least 99.99% mol, at least 99.9925% mol, at least 99.995% mol, at least 99.9975% mol, at least 99.999% mol, at least 99.9999% mol, or at least 99.99999% mol.

In some embodiments according to the disclosure, either alone or together with any one or more of the aforementioned and/or after-mentioned embodiments, the contacting, separating, or administering are performed with at least one selected from the group consisting of: a power injector, a syringe, an inline filter housing, and a manual bolus injector.

In some embodiments according to the disclosure, either alone or together with any one or more of the aforementioned and/or after-mentioned embodiments, the contacting is performed for at least 5 minutes.

In some embodiments according to the disclosure, either alone or together with any one or more of the aforementioned and/or after-mentioned embodiments, the method further includes agitating the solution during the contacting.

In some embodiments according to the disclosure, either alone or together with any one or more of the aforementioned and/or after-mentioned embodiments, the GBCA is selected from the group consisting of: gadoterate, gadobutrol, gadoteridol, gadopiclenol, and gadoquatrane.

In some embodiments according to the disclosure, either alone or together with any one or more of the aforementioned and/or after-mentioned embodiments, the agent is a trivalent-cation selective exchange medium.

In some embodiments according to the disclosure, either alone or together with any one or more of the aforementioned and/or after-mentioned embodiments, the method further includes freezing and storing the solution and the agent at a temperature of less than 5 °C below a freezing point of the GBCA, and thawing the frozen solution and agent to room temperature prior to the administering.

In some embodiments according to the disclosure, either alone or together with any one or more of the aforementioned and/or after-mentioned embodiments, the solution and the agent are stored and frozen prior to the contacting, so that upon thawing, the solution contacts the agent.

In some embodiments according to the disclosure, either alone or together with any one or more of the aforementioned and/or after-mentioned embodiments, the solution is frozen and stored with a dry agent prior to the contacting, so that upon thawing, the solution contacts the agent.

In some embodiments according to the disclosure, either alone or together with any one or more of the aforementioned and/or after-mentioned embodiments, the solution and the agent are stored after the contacting.

In some embodiments according to the disclosure, either alone or together with any one or more of the aforementioned and/or after-mentioned embodiments, the method further includes freezing the solution at a temperature of less than 5 °C below a freezing point of the GBCA after the separating, storing the frozen solution at the freezing temperature, and thawing the frozen solution and agent to room temperature prior to the administering.

In some embodiments according to the disclosure, either alone or together with any one or more of the aforementioned and/or after-mentioned embodiments, the solution is thawed from a frozen state prior to the contacting.

In some embodiments according to the disclosure, either alone or together with any one or more of the aforementioned and/or after-mentioned embodiments, the method further includes drying the solution to remove free water after the separating to yield a dry GBCA, the drying being performed by at least one selected from the group consisting of: lyophilization, flash evaporation, crystallization and drying, and vacuum spray drying, and reconstituting the dry GBCA in a pharmaceutically acceptable aqueous solution prior to the administering.

In certain embodiments, the present disclosure provides a dry composition comprising a macrocyclic gadolinium-based contrast agent (GBCA) in which drying methods can be continued beyond the removal of free water, resulting in a composition with a reduced amount of bound water. In some embodiments, the dry composition comprises less than 5 wt %, 4 wt %, 3 wt %, 2 wt %, 1 wt %, or 0.5 wt % bound water. Such compositions represent an end state of drying and are provided as solid materials suitable for reconstitution into pharmaceutically acceptable injectable solutions. In some embodiments, a selective exchange medium in a dry state is combined with the dry GBCA composition either prior to storage or upon reconstitution of the dry GBCA composition. Without wishing to be bound by theory, it is believed that upon reconstitution, the macrocyclic gadolinium complex may preferentially coordinate with a single water molecule in the inner coordination sphere, thereby reducing or delaying redistribution among alternative hydrated coordination states.

In some embodiments, the macrocyclic gadolinium-based contrast agent is selected from gadoterate, gadobutrol, gadoteridol, gadopiclenol, and gadoquatrane. In particular embodiments, the dry composition comprises gadoterate meglumine having less than about 1 wt % bound water.

In some embodiments according to the disclosure, either alone or together with any one or more of the aforementioned and/or after-mentioned embodiments, the method further includes contacting the reconstituted solution with a second agent configured to induce dechelation of Gd ions from partially coordinated complexes of the GBCA and bind dissociated Gd ions, and separating the second agent from the reconstituted solution prior to the administering.

In some embodiments according to the disclosure, either alone or together with any one or more of the aforementioned and/or after-mentioned embodiments, the method further includes preparing the solution by combining a dried GBCA with a pharmaceutically acceptable aqueous solution.

A method according to the present disclosure includes contacting a solution comprising a macrocyclic chelated gadolinium-based contrast agent (GBCA) with an agent configured to induce dechelation of Gd from partially coordinated complexes of the GBCA and bind dissociated Gd ions, and processing the solution while the solution comprises a decreased proportion of partially coordinated complexes of the GBCA relative to the solution prior to the contacting. The processing yields a storable GBCA composition in which, during storage, the proportion of the partially coordinated complexes after the contacting remains below the proportion of the partially coordinated complexes prior to the contacting.

In some embodiments according to the disclosure, either alone or together with any one or more of the aforementioned and/or after-mentioned embodiments, the method further includes removing the agent prior to the processing.

In some embodiments according to the disclosure, either alone or together with any one or more of the aforementioned and/or after-mentioned embodiments, the method further includes storing the agent or a second agent configured to induce dechelation of Gd ion from partially coordinated complexes of the GBCA and bind dissociated Gd ions with the storable GBCA composition.

In some embodiments according to the disclosure, either alone or together with any one or more of the aforementioned and/or after-mentioned embodiments, the processing is at least one selected from the group consisting of: freezing to immobilize free water, phase separation to separate the GBCA from free water, or drying to remove free water.

In some embodiments according to the disclosure, either alone or together with any one or more of the aforementioned and/or after-mentioned embodiments, the drying is performed to remove free water by at least one selected from the group consisting of: lyophilization, flash evaporation, crystallization and drying, and vacuum spray drying.

In some embodiments according to the disclosure, either alone or together with any one or more of the aforementioned and/or after-mentioned embodiments, the processing includes vacuum spray drying the macrocyclic chelated GBCA complex at 40-60 C° to yield a dry product having a free water moisture level of 1% to 5%, 2% to 4%, or 1% to 3%, and the method further includes packaging the dry product in a sealed container under dry inert gas or dry air having a humidity of at most 30%.

In some embodiments according to the disclosure, either alone or together with any one or more of the aforementioned and/or after-mentioned embodiments, the processing includes freezing the macrocyclic chelated GBCA solution in a lyophilization chamber to a temperature of less than 5 °C below its freezing point, applying a vacuum to the lyophilization chamber to maintain a pressure, sublimating free water from the frozen macrocyclic complex until a free water content is 1% to 5% to yield the storable GBCA composition, and sealing a container with the storable GBCA composition.

In some embodiments according to the disclosure, either alone or together with any one or more of the aforementioned and/or after-mentioned embodiments, the processing includes feeding the solution to a flash evaporator through an atomizing nozzle into a stream of nitrogen heated to 120 to 185 °C to yield the storable GBCA composition.

In some embodiments according to the disclosure, either alone or together with any one or more of the aforementioned and/or after-mentioned embodiments, the method further includes packaging the solid product in a container under dry inert gas or air having a humidity of at most 30%, and sealing the container.

In some embodiments according to the disclosure, either alone or together with any one or more of the aforementioned and/or after-mentioned embodiments, the storable GBCA composition has a moisture level of 1% to 5%, 2% to 4%, or 1% to 3%.

In some embodiments according to the disclosure, either alone or together with any one or more of the aforementioned and/or after-mentioned embodiments, the processing includes precipitating the macrocyclic chelated GBCA from solution, and drying macrocyclic chelated GBCA to remove free water.

In some embodiments according to the disclosure, either alone or together with any one or more of the aforementioned and/or after-mentioned embodiments, the processing includes freezing the solution, and storing the frozen solution at a temperature of less than −40 °C.

In some embodiments according to the disclosure, either alone or together with any one or more of the aforementioned and/or after-mentioned embodiments, the agent is frozen with the solution.

In some embodiments according to the disclosure, either alone or together with any one or more of the aforementioned and/or after-mentioned embodiments, the agent is further configured to isolate and remove partially coordinated complexes of the GBCA in the solution.

In some embodiments according to the disclosure, either alone or together with any one or more of the aforementioned and/or after-mentioned embodiments, the agent is further configured to isolate and remove free macrocyclic chelate in the solution.

In some embodiments according to the disclosure, either alone or together with any one or more of the aforementioned and/or after-mentioned embodiments, the agent is further configured to isolate and remove partially coordinated complexes of the GBCA in the solution and free macrocyclic chelate in the solution.

In some embodiments according to the disclosure, either alone or together with any one or more of the aforementioned and/or after-mentioned embodiments, the macrocyclic chelated GBCA is selected from the group consisting of: gadoterate, gadobutrol, gadoteridol, gadopiclenol, and gadoquatrane.

A method according to the present disclosure includes administering to a subject in need thereof a diagnostically effective amount of a macrocyclic GBCA in a pharmaceutically acceptable aqueous solution, wherein, based on total GBCA complexes present, at least 99% mol are fully coordinated complexes in which Gd ion is bound to all coordination sites of a macrocyclic chelator of the GBCA, and at most 1% mol are partially coordinated complexes in which Gd ion is bound to fewer than all coordination sites of the macrocyclic chelator.

In some embodiments according to the disclosure, either alone or together with any one or more of the aforementioned and/or after-mentioned embodiments, where at least 99% mol of total GBCA complexes are fully coordinated complexes in which Gd ion is bound to all coordination sites of the macrocyclic chelator, and not more than 1% mol are partially coordinated complexes in which Gd ion is bound to fewer than all coordination sites of the macrocyclic chelator, the proportion of partially coordinated complexes is not more than 1% mol, less than 0.9% mol, less than 0.8% mol, less than 0.7% mol, less than 0.6% mol, less than 0.5% mol, less than 0.4% mol, less than 0.3% mol, less than 0.2% mol, less than 0.1% mol, less than 0.075% mol, less than 0.05% mol, less than 0.025% mol, less than 0.01% mol, less than 0.0075% mol, less than 0.005% mol, less than 0.0025% mol, less than 0.001% mol, less than 0.0001% mol, or less than 0.00001% mol. In these embodiments, the proportion of fully coordinated complexes is at least 99% mol, at least 99.1% mol, at least 99.2% mol, at least 99.3% mol, at least 99.4% mol, at least 99.5% mol, at least 99.6% mol, at least 99.7% mol, at least 99.8% mol, at least 99.9% mol, at least 99.925% mol, at least 99.95% mol, at least 99.975% mol, at least 99.99% mol, at least 99.9925% mol, at least 99.995% mol, at least 99.9975% mol, at least 99.999% mol, at least 99.9999% mol, or at least 99.99999% mol.

In some embodiments according to the disclosure, either alone or together with any one or more of the aforementioned and/or after-mentioned embodiments, the GBCA is selected from the group consisting of: gadoterate, gadobutrol, gadoteridol, gadopiclenol, and gadoquatrane.

In some embodiments according to the disclosure, either alone or together with any one or more of the aforementioned and/or after-mentioned embodiments, the GBCA is gadoterate, and the composition has a 2 hour conditional partial coordination equilibrium constant of at least 6.9 as determined by Resin-UV Comparative Protocol.

In some embodiments according to the disclosure, either alone or together with any one or more of the aforementioned and/or after-mentioned embodiments, the GBCA is gadobutrol and the composition has a 2 hour conditional partial coordination equilibrium constant of at least 5.4 as determined by Resin-UV Comparative Protocol.

A composition according to the present disclosure includes a pharmaceutically acceptable API of a gadolinium-based contrast agent (GBCA), with at least 99% mol of total GBCA complexes being fully coordinated complexes in which Gd ion is bound to all coordination sites of the macrocyclic chelator, and not more than 1% mol being partially coordinated complexes in which Gd ion is bound to fewer than all coordination sites of the macrocyclic chelator.

In some embodiments according to the disclosure, either alone or together with any one or more of the aforementioned and/or after-mentioned embodiments, where at least 99% mol of total GBCA complexes are fully coordinated complexes in which Gd ion is bound to all coordination sites of the macrocyclic chelator, and not more than 1% mol are partially coordinated complexes in which Gd ion is bound to fewer than all coordination sites of the macrocyclic chelator, the proportion of partially coordinated complexes is not more than 1% mol, less than 0.9% mol, less than 0.8% mol, less than 0.7% mol, less than 0.6% mol, less than 0.5% mol, less than 0.4% mol, less than 0.3% mol, less than 0.2% mol, less than 0.1% mol, less than 0.075% mol, less than 0.05% mol, less than 0.025% mol, less than 0.01% mol, less than 0.0075% mol, less than 0.005% mol, less than 0.0025% mol, less than 0.001% mol, less than 0.0001% mol, or less than 0.00001% mol. In these embodiments, the proportion of fully coordinated complexes is at least 99% mol, at least 99.1% mol, at least 99.2% mol, at least 99.3% mol, at least 99.4% mol, at least 99.5% mol, at least 99.6% mol, at least 99.7% mol, at least 99.8% mol, at least 99.9% mol, at least 99.925% mol, at least 99.95% mol, at least 99.975% mol, at least 99.99% mol, at least 99.9925% mol, at least 99.995% mol, at least 99.9975% mol, at least 99.999% mol, at least 99.9999% mol, or at least 99.99999% mol.

In some embodiments according to the disclosure, either alone or together with any one or more of the aforementioned and/or after-mentioned embodiments, the GBCA is selected from the group consisting of: gadoterate, gadobutrol, gadoteridol, gadopiclenol, and gadoquatrane.

In some embodiments according to the disclosure, either alone or together with any one or more of the aforementioned and/or after-mentioned embodiments, the GBCA is gadoterate and the composition has a 2 hour conditional partial coordination equilibrium constant of at least 6.9 as determined by Resin-UV Comparative Protocol.

In some embodiments according to the disclosure, either alone or together with any one or more of the aforementioned and/or after-mentioned embodiments, the GBCA is gadobutrol and the composition has a 2 hour conditional partial coordination equilibrium constant of at least 5.4 as determined by Resin-UV Comparative Protocol.

In some embodiments according to the disclosure, either alone or together with any one or more of the aforementioned and/or after-mentioned embodiments, the composition is in solution and the solution is frozen.

In some embodiments according to the disclosure, either alone or together with any one or more of the aforementioned and/or after-mentioned embodiments, the API has a free water content of at most 5%.

A composition according to the present disclosure includes a gadolinium-based contrast agent (GBCA) in a pharmaceutically acceptable aqueous solution, and the composition has a 2 hour conditional partial coordination equilibrium constant of at least 5.4 as determined by Resin-UV Comparative Protocol.

In some embodiments according to the disclosure, either alone or together with any one or more of the aforementioned and/or after-mentioned embodiments, the GBCA is selected from the group consisting of: gadoterate, gadobutrol, gadoteridol, gadopiclenol, and gadoquatrane.

In some embodiments according to the disclosure, either alone or together with any one or more of the aforementioned and/or after-mentioned embodiments, the GBCA is gadoterate and the composition has a 2 hour conditional partial coordination equilibrium constant of at least 6.9 as determined by Resin-UV Comparative Protocol.

In some embodiments according to the disclosure, either alone or together with any one or more of the aforementioned and/or after-mentioned embodiments, the GBCA is gadobutrol and the composition has a 2 hour conditional partial coordination equilibrium constant of at least 5.4 as determined by Resin-UV Comparative Protocol.

In some embodiments according to the disclosure, either alone or together with any one or more of the aforementioned and/or after-mentioned embodiments, at least 99% mol of total GBCA complexes are fully coordinated complexes in which Gd ion is bound to all coordination sites of the macrocyclic chelator, and not more than 1% mol are partially coordinated complexes in which Gd ion is bound to fewer than all coordination sites of the macrocyclic chelator.

In some embodiments according to the disclosure, either alone or together with any one or more of the aforementioned and/or after-mentioned embodiments, where at least 99% mol of total GBCA complexes are fully coordinated complexes in which Gd ion is bound to all coordination sites of the macrocyclic chelator, and not more than 1% mol are partially coordinated complexes in which Gd ion is bound to fewer than all coordination sites of the macrocyclic chelator, the proportion of partially coordinated complexes is not more than 1% mol, less than 0.9% mol, less than 0.8% mol, less than 0.7% mol, less than 0.6% mol, less than 0.5% mol, less than 0.4% mol, less than 0.3% mol, less than 0.2% mol, less than 0.1% mol, less than 0.075% mol, less than 0.05% mol, less than 0.025% mol, less than 0.01% mol, less than 0.0075% mol, less than 0.005% mol, less than 0.0025% mol, less than 0.001% mol, less than 0.0001% mol, or less than 0.00001% mol. In these embodiments, the proportion of fully coordinated complexes is at least 99% mol, at least 99.1% mol, at least 99.2% mol, at least 99.3% mol, at least 99.4% mol, at least 99.5% mol, at least 99.6% mol, at least 99.7% mol, at least 99.8% mol, at least 99.9% mol, at least 99.925% mol, at least 99.95% mol, at least 99.975% mol, at least 99.99% mol, at least 99.9925% mol, at least 99.995% mol, at least 99.9975% mol, at least 99.999% mol, at least 99.9999% mol, or at least 99.99999% mol.

A kit according to the present disclosure includes a macrocyclic chelated gadolinium-based contrast agent (GBCA) in a pharmaceutically acceptable aqueous solution disposed in a first container, and an agent configured to induce dechelation of Gd ions from partially coordinated complexes of the macrocyclic chelated GBCA complex and bind dissociated Gd ions.

In some embodiments according to the disclosure, either alone or together with any one or more of the aforementioned and/or after-mentioned embodiments, the GBCA is selected from the group consisting of: gadoterate, gadobutrol, gadoteridol, gadopiclenol, and gadoquatrane.

In some embodiments according to the disclosure, either alone or together with any one or more of the aforementioned and/or after-mentioned embodiments, the agent is a cation exchange resin or a membrane.

In some embodiments according to the disclosure, either alone or together with any one or more of the aforementioned and/or after-mentioned embodiments, the selective exchange medium can be provided in a wet (hydrated) state or in a dry state. In embodiments in which the selective exchange medium is provided in a wet state, the medium is already hydrated and does not substantially absorb additional liquid upon contact with an aqueous composition. In embodiments in which the selective exchange medium is provided in a dry state, the medium can absorb and retain liquid upon hydration. As a result, the volume of liquid present and the effective concentration of the macrocyclic gadolinium-based contrast agent in contact with the selective exchange medium may differ depending on whether the selective exchange medium is initially wet or dry. Such differences may be taken into account when selecting relative amounts of the selective exchange medium and liquid, while maintaining interchangeability of wet and dry selective exchange media embodiments. The wet or dry state of the selective exchange medium does not alter the ability of the selective exchange medium to bind dissociated gadolinium ions once hydrated or activated. It will also be appreciated that a wet or hydrated selective exchange medium can be dried or frozen using the methods discussed herein, and also subsequently rehydrated or thawed. Likewise, a selective exchange medium in a dry state can be frozen or hydrated.

In some embodiments according to the disclosure, features described in connection with any embodiment are combinable with features of any other embodiment unless technically incompatible.

The above-described and other features and advantages of the present disclosure will be appreciated and understood by those skilled in the art from the following detailed description, drawings, and appended claims.

### BRIEF DESCRIPTION OF THE DRAWINGS

FIG. 1 illustrates an embodiment of a syringe comprising a selective exchange medium disposed within the barrel for contacting a GBCA solution before administration.

FIG. 2 illustrates an embodiment of a dual-chamber syringe in which a GBCA solution and a selective exchange medium are mixed in situ before administration.

FIG. 3 illustrates an embodiment of an inline filter assembly containing a selective exchange medium positioned to contact a GBCA solution flowing through the device.

FIG. 4 illustrates an embodiment of a microchannel contactor including one or more flow paths containing a selective exchange medium for contacting a GBCA solution.

FIG. 5 illustrates an embodiment of a bulb-actuated device having a treatment chamber containing a selective exchange medium for contacting a GBCA solution or a reconstituted GBCA formulation before administration.

FIG. 6 illustrates an embodiment of a power injector system incorporating a treatment module containing a selective exchange medium for contacting a GBCA solution drawn from a bulk vial before administration.

FIGS. 7 and 8 show timeseries testing for fully and partially coordinated Gd-DOTA complexes according to the disclosure.

FIGS. 9, 10, and 11 show timeseries testing for fully and partially coordinated Gd-DOTA complexes, as disclosed.

FIG. 12 shows a timeseries of testing for fully and partially coordinated Gd-DOTA complexes with freezing, as disclosed.

FIG. 13 shows a timeseries testing for fully and partially coordinated complexes of Gd-DOTA with freezing plus selective exchange resin according to the disclosure

FIG. 14 shows a timeseries of testing for fully and partially coordinated complexes of Gd-DOTA with freezing plus selective exchange resin, according to the disclosure.

FIG. 15 shows a timeseries of testing for fully and partially coordinated complexes of Gd-DOTA with freezing plus selective exchange resin, according to the disclosure.

FIG. 16 shows a timeseries of testing for fully and partially coordinated complexes of Gd-DOTA with freezing plus selective exchange resin, according to the disclosure.

FIG. 17 shows a timeseries test for fully and partially coordinated Gd-DOTA complexes with freezing alone, as described in the disclosure.

FIG. 18 shows a timeseries of testing for fully and partially coordinated Gd-DOTA complexes using the mix-on-demand method of the disclosure.

FIG. 19 shows a timeseries of testing for fully and partially coordinated Gd-DOTA complexes using the mix-on-demand method of the disclosure.

FIG. 20 shows a timeseries of testing for fully and partially coordinated gadobutrol complexes.

FIG. 21 shows another timeseries of testing for fully and partially coordinated gadobutrol complexes.

FIG. 22 shows another timeseries of testing for fully and partially coordinated Gd-DOTA complexes using the mix-on-demand method of the disclosure

FIG. 23 shows a timeseries testing for fully and partially coordinated Gd-DOTA complexes according to the disclosure.

FIG. 24 shows a timeseries testing for fully and partially coordinated gadopiclinol complexes without the exchange media treatment of the present disclosure.

FIG. 25 shows a timeseries testing for fully and partially coordinated gadopiclinol complexes according to the disclosure.

FIGS. 26, 27, 28, 29, and 30 show spray-drying test data.

### DETAILED DESCRIPTION OF THE DISCLOSURE

The present disclosure provides various methods for minimizing or delaying the formation of partially coordinated complexes in macrocyclic chelated GBCA complexes used in MRIs, thus prolonging chelate integrity.

The macrocyclic chelated GBCA complexes can either be obtained and provided or synthesized.

A first set of example methods according to the disclosure is referred to as a mix-on-demand method.

For example, one such method includes contacting a solution of a macrocyclic chelated GBCA with an agent configured to induce dechelation of Gd ions from partially coordinated complexes of the GBCA and bind dissociated Gd ions, separating the agent from the solution, administering the solution to a patient while the solution comprises a decreased proportion of partially coordinated complexes of the GBCA relative to the solution before the contacting. The agent can be a selective exchange medium

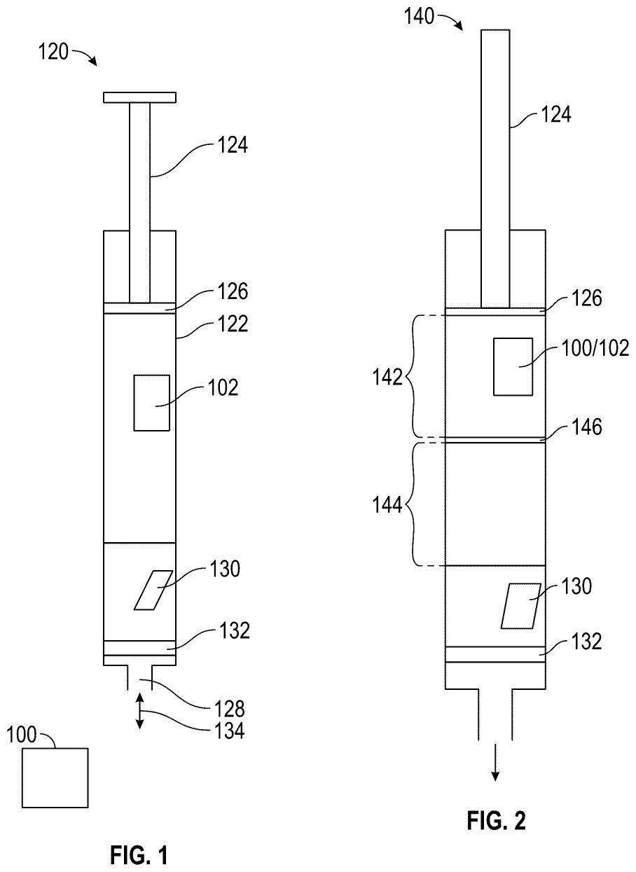

Referring to FIG. 1, such a method can be performed, for example, using a syringe 120. Syringe 120 includes a barrel 122, a plunger 124 with a stopper 126, and a port 128.

Selective exchange medium 130 is disposed within barrel 122 between port 128 and stopper 126. Selective exchange medium 130 can be a cation exchange resin in bead, monolith, or porous matrix form, an ion exchange membrane, a functionalized polymer, a chelating polymer, a sulfonated or carboxylated surface, a mixed-bed ion exchanger, combinations thereof, or an equivalent medium capable of inducing dechelation of dissociated Gd ions from partially coordinated complexes and binding the dissociated ions, including any such selective exchange medium disclosed herein.

Selective exchange medium 130 can be retained between screens or frits, immobilized on or within a carrier, such as a polymer film, captured in a cartridge, or embedded in a porous scaffold to prevent discharge through port 128 during administration. There can also be a filter 132 disposed at port 128. Filter 132 can, for example, be a 0.2 to 0.5 micron filter.

A GBCA solution 100 can be drawn through port 128 into barrel 122 as indicated by arrow 134 to contact selective exchange medium 130 for a residence time sufficient to decrease the fraction of partially coordinated complexes and increase fully coordinated complexes. Optional valves and user actuators can control filling and dispensing, but are not shown.

As an alternative, a dry GBCA 102 can be positioned in the syringe, and water can be drawn into the syringe to form a GBCA solution.

Syringe 120 can be gently inverted or rotated end-over-end approximately 10 to 20 times, or maintained in motion or agitated for about 1 to 5 minutes, or more, to enhance contact between the GBCA solution 100 and selective exchange medium 130. This mixing facilitates attainment of the desired equilibrium composition in which the proportion of fully coordinated complexes increases relative to partially coordinated complexes. Thereafter, the GBCA solution can be administered to a patient.

Referring to FIG. 2, such a method can be performed, for example, using a dual-chamber syringe 140. Dual-chamber syringe 140 includes a barrel 122, a plunger 124 with a stopper 126, and a port 128. Barrel 122 defines a first chamber 142 containing a GBCA solution 100, and a second chamber 144 containing a selective exchange medium 130. The two chambers, first chamber 142 and second chamber 144 are separated by a frangible seal 146, bypass, or other movable barrier that can be displaced, punctured, or ruptured to permit mixing or flow between the chambers.

Selective exchange medium 130 can be a cation exchange resin in bead, monolith, or porous matrix form, an ion exchange membrane, a functionalized polymer, a chelating polymer, a sulfonated or carboxylated surface, a mixed-bed ion exchanger, combinations thereof, or an equivalent medium capable of inducing dechelation of dissociated Gd ions from partially coordinated complexes and binding the dissociated ions, including any such selective exchange medium disclosed herein.

Selective exchange medium 130 can be retained between screens or frits, immobilized on or within a carrier, such as a polymer film or porous scaffold, or captured within a cartridge to prevent discharge through port 128 during administration. There can also be a filter 132 disposed at port 128. Filter 132, for example, can be a 0.2 to 0.5 micron filter that prevents the passage of any residual media or particulates.

When use is desired, actuation of plunger 124 or release of frangible seal 146 allows GBCA solution 100 in first chamber 142 to mix with or flow into second chamber 144, which contains selective exchange medium 130. Syringe 140 can be gently inverted or rotated end-over-end approximately 10 to 20 times or maintained in motion or agitated for about 1 to 5 minutes, or more, to enhance contact between GBCA solution 100 and selective exchange medium 130. This contacting step decreases the fraction of partially coordinated complexes and increases the fraction of fully coordinated complexes. Optional valves and user actuators can control mixing or flow between the chambers, but are not shown.

As an alternative, a dry GBCA 102 can be disposed within one chamber, for example, first chamber 142, and water or another carrier liquid can be disposed in the other chamber, for example, second chamber 144, with the selective exchange medium 130. Upon activation, the water and GBCA combine and contact selective exchange medium 130, forming the GBCA solution and completing the exchange process. Thereafter, the treated GBCA can be administered to a patient.

Examples of equivalent constructions include, but are not limited to, multi-segment or nested chambers, coaxial barrels, or staged media zones providing sequential exchange media treatment. Such a construction can be two separate vials, where the GBCA solution is mixed in the clinic for 1 to 5 minutes, or more, and the required dose is withdrawn through a filter to remove the selective exchange medium 130 before administering to a patient.

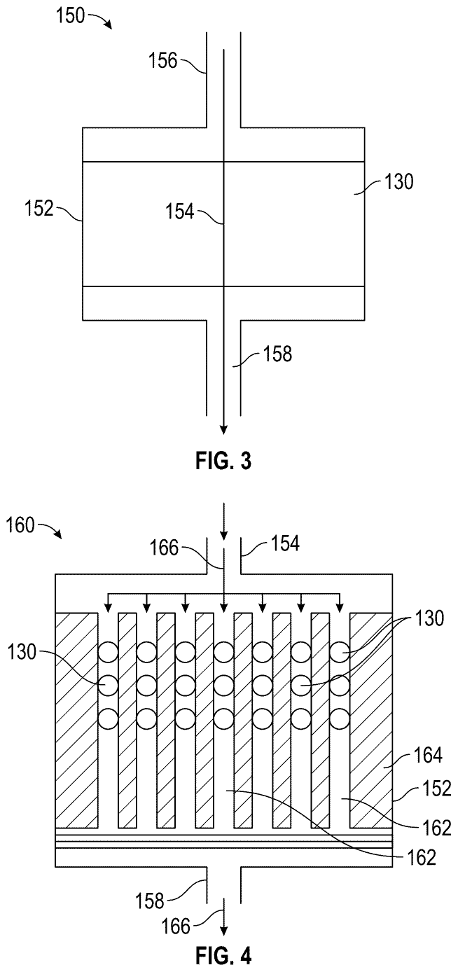

Referring to FIG. 3, such a method can be performed, for example, using an inline filter assembly 150. Inline filter assembly 150 includes a housing 152 defining a flow path 154 extending between an inlet port 156 and an outlet port 158. Disposed within housing 152, across flow path 154, is a selective exchange medium 130 configured to contact a solution comprising a GBCA 100.

Selective exchange medium 130 can be a cation exchange resin in bead, monolith, or porous matrix form, an ion exchange membrane, a functionalized polymer, a chelating polymer, a sulfonated or carboxylated surface, a mixed-bed ion exchanger, combinations thereof, or an equivalent medium capable of inducing dechelation of dissociated Gd ions from partially coordinated complexes and binding the dissociated ions, including any such selective exchange medium disclosed herein.

Selective exchange medium 130 can be configured as a membrane element, a packed-bed cartridge, or a composite structure combining a membrane and a resin component. Selective exchange medium 130 can be retained within housing 152 by screens or frits, by welded or molded supports, or by a removable cartridge insert, thereby preventing discharge through outlet port 158 during administration. There can also be a filter disposed at outlet port 158. Filter 132, for example, can be a 0.2 to 0.5 micron filter positioned downstream of selective exchange medium 130 to remove residual particulates.

During operation, the GBCA solution 100 is directed through inlet port 156 and along flow path 154 to contact selective exchange medium 130 for a residence time sufficient to decrease the fraction of partially coordinated complexes and increase the fraction of fully coordinated complexes. The contacting step induces dechelation of dissociated Gd ions from weaker complexes and their binding by selective exchange medium 130. The treated solution exits through outlet port 158 for administration.

Optional valves, bypass lines, and user actuators can control flow through the assembly. Inline filter assembly 150 can be incorporated into an IV line, a power injector circuit, or a prefilled delivery set and can include additional features, such as valves or flow restrictors, to maintain consistent operation. Examples of equivalent embodiments include, but are not limited to, multi-stage cartridges, parallel flow modules, or hollow-fiber membranes, provided that the selective exchange medium 130 contacts the GBCA solution and is separated therefrom before administration.

Referring to FIG. 4, such a method can be performed, for example, using a microchannel assembly 160. Microchannel assembly 160 includes a housing 152 defining a flow path 166 extending between an inlet port 156 and an outlet port 158. Microchannel assembly 160 includes one or more microchannels 162 defined within a substrate 164 or between stacked layers of a laminated or molded structure in housing 152. Each microchannel 162 defines a flow path 166 through which a solution comprising a GBCA 100 passes and contacts a selective exchange medium 130.

Selective exchange medium 130 can be disposed within one or more regions of the microchannels 162, for example, as a packed-bed region, a monolithic insert, a porous matrix, or a functionalized internal surface coating. Selective exchange medium 130 can be a cation exchange resin, an ion exchange membrane, a functionalized polymer, a chelating polymer, a sulfonated or carboxylated surface, a mixed-bed ion exchanger, combinations thereof, or an equivalent medium capable of inducing dechelation of dissociated Gd ions from partially coordinated complexes and binding the dissociated ions, including any such selective exchange medium disclosed herein.

The selective exchange medium 130 can be retained within the microchannels 162 by porous frits, microscreens, flow constrictions, or selective barriers that permit fluid passage while preventing the medium's movement. The microchannels 162 can have rectangular, circular, serpentine, spiral, or branching geometries to provide a desired contact time and pressure drop.

Examples of equivalent constructions include, but are not limited to, etched, molded, or additive-manufactured components fabricated from polymeric, glass, ceramic, or metal materials compatible with the GBCA solution.

In operation, the GBCA solution 100 flows through microchannels 162 to contact the selective exchange medium 130 for a residence time sufficient to decrease the fraction of partially coordinated complexes and increase the fraction of fully coordinated complexes. The contacting step induces dechelation of dissociated Gd ions from weaker complexes and binding of the ions by the selective exchange medium 130. The treated solution exits the microchannels 162 and is directed to outlet port 158 for administration.

Optional flow distributors, valves, or actuators can regulate flow through the microchannels. Microchannel assembly 160 can be configured as a standalone cartridge, an inline module, or an integrated component of a syringe, infusion set, or power injector circuit. Examples of other equivalent constructions include, but are not limited to, arrays of parallel microchannels, multi-zone devices with varying media types or exchange capacities, and reversible flow designs that provide extended or repeated contact between selective exchange medium 130 and GBCA solution 100.

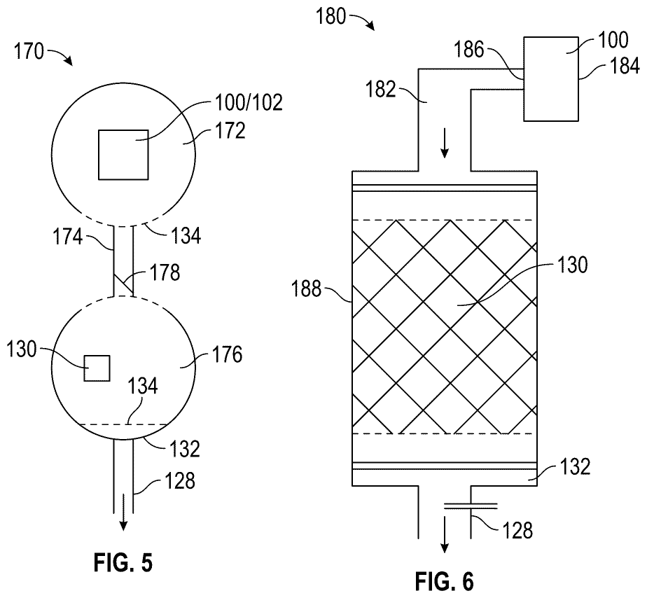

Referring to FIG. 5, such a method can be performed, for example, using a bulb-actuated device 170. Bulb-actuated device 170 includes a bulb 172 and a treatment chamber 176 connected by a conduit 174, with an optional one-way valve 178 therebetween. Bulb 172 can be flexible or resilient to permit manual compression and release. Treatment chamber 176 contains a selective exchange medium 130 configured to contact a GBCA 100 in liquid or reconstituted form. Each of bulb 172 and treatment chamber 176 can have a pressure-sensitive member 134.

Selective exchange medium 130 can be a cation exchange resin in bead, monolith, or porous matrix form, an ion exchange membrane, a functionalized polymer, a chelating polymer, a sulfonated or carboxylated surface, a mixed-bed ion exchanger, combinations thereof, or an equivalent medium capable of inducing dechelation of dissociated Gd ions from partially coordinated complexes and binding the dissociated ions, including any such selective exchange medium disclosed herein. Selective exchange medium 130 can be retained within treatment chamber 176 by screens or frits, immobilized within a porous scaffold, or contained in a replaceable cartridge to prevent discharge during administration. A filter 132, for example, a 0.2 to 0.5 micron filter, can be positioned downstream of treatment chamber 176 to capture residual particulates.

In a first mode of operation, a solution comprising a GBCA 100 is positioned within bulb 172. Upon compression of bulb 172, the solution is forced through conduit 174 and valve 178 into treatment chamber 176, where it contacts selective exchange medium 130. The contacting step continues for a residence time sufficient to decrease the fraction of partially coordinated complexes and increase the fraction of fully coordinated complexes. The treated solution is then expelled through a port 128 for administration.

In a second mode of operation, bulb 172 contains water 194 or another carrier liquid, and treatment chamber 176 contains dry GBCA 102 together with selective exchange medium 130. Compression of bulb 172 can transfer water 194 into treatment chamber 176, hydrating the dry GBCA 102 and bringing it into contact with selective exchange medium 130. The contacting step induces dechelation of dissociated Gd ions from partially coordinated complexes and binding of the ions by the selective exchange medium 130.

In both modes of operation, the mixture can be gently agitated or maintained in motion for about 5 minutes to complete the exchange process. The treated GBCA solution can then be expelled through port 128 for administration.

Optional valves and actuators can control flow between bulb 172 and treatment chamber 176. Examples of other equivalent constructions include, but are not limited to, bellows-type pumps, compressible reservoirs, and multi-chamber squeeze devices in which the selective exchange medium 130 can be contacted by GBCA solution 100 and retained before delivery.

Optionally, the syringes, vials, or devices discussed above can be frozen and then thawed prior to administration as a combination method.

A larger batch preparation method involves, as an example, holding a master batch of macrocyclic chelated GBCA complexes at −10 °C to room temperature for up to 8 hours before disposal. As an example, a 200 mL master batch can be prepared and shaken for 1 to 20 minutes, then filtered prior to use.

Referring to FIG. 6, such a method can be performed, for example, using a power injector system 180 configured for multi-dose operation. Power injector system 180 includes a vial 184 containing a GBCA solution 100, a vial interface 186, and a fluid line 182 extending from vial 184 to an injector head or delivery outlet, port 128. Vial 184 can be a bulk vial, for example. Disposed along fluid line 182 is a treatment chamber 188 containing a selective exchange medium 130 configured to contact the GBCA solution 100 during transfer or administration.

Selective exchange medium 130 can be a cation exchange resin in bead, monolith, or porous matrix form, an ion exchange membrane, a functionalized polymer, a chelating polymer, a sulfonated or carboxylated surface, a mixed-bed ion exchanger, combinations thereof, or an equivalent medium capable of inducing dechelation of dissociated Gd ions from partially coordinated complexes and binding the dissociated ions, including any such selective exchange medium disclosed herein. Selective exchange medium 130 can be incorporated as a membrane element, a packed-bed cartridge, or a hybrid structure within treatment chamber 188, and can be retained by screens or frits or embedded within a porous scaffold to prevent discharge through the delivery outlet, port 128. A filter 132, for example, a 0.2 to 0.5 micron filter, can be positioned downstream of treatment chamber 188.

During operation, GBCA solution 100 is drawn from vial 184 through vial interface 186 into fluid line 182 and through treatment chamber 188, where it contacts selective exchange medium 130. The contacting step induces dechelation of dissociated Gd ions from partially coordinated complexes and binding of the ions by the selective exchange medium 130, decreasing the fraction of partially coordinated complexes and increasing fully coordinated complexes prior to administration. The treated GBCA solution then can exit from treatment chamber 188 and be delivered through port 128 to a patient line.

Power injector system 180 can include one or more valves, vents, pressure sensors, and flow controllers to regulate fluid movement and maintain consistent contact time. Selective exchange medium 130 can be positioned within a disposable cartridge, a replaceable filter assembly, or an integrated pump head.

Examples of other equivalent constructions include, but are not limited to, serial or parallel multi-cartridge configurations, modular inline filters, and disposable injector sets, provided that the GBCA solution contacts the selective exchange medium 130 and is separated therefrom before administration.

Filtering in the general sense means separating the selective exchange medium from the macrocyclic chelated GBCA complexes so that the selective exchange medium is not injected into a patient, and can be accomplished by other known methods not explicitly described above.

A second set of example methods according to the disclosure is referred to as freezing methods.

Macrocyclic chelated GBCA complexes can be frozen immediately after synthesis, optionally together with a selective exchange medium. Here, the term immediately means prior to the formation or reformation of more than 1%, 0.9%, 0.8%, 0.7%, 0.6%, 0.5%, 0.4%, 0.3%, or 0.2% partially coordinated complexes.

Macrocyclic chelated GBCA complexes can also be frozen immediately after a first exchange media treatment, optionally together with the first selective exchange medium or with a second fresh selective exchange medium and the first selective exchange medium removed. Here, the term immediately means prior to the formation or reformation of more than 1%, 0.9%, 0.8%, 0.7%, 0.6%, 0.5%, 0.4%, 0.3%, or 0.2% partially coordinated complexes.

Without wishing to be bound by theory, it is believed that the freezing process deactivates the selective exchange medium, and the subsequent thawing process reactivates it. In this context, deactivation means that the inducing by the selective exchange medium of dechelation of Gd ion from partially coordinated complexes of the macrocyclic chelated GBCA complex and binding dissociated Gd ions is slowed relative to above freezing temperatures, preferably halted. Activation means that the inducing by the selective exchange medium of dechelation of Gd from partially coordinated complexes of the macrocyclic chelated GBCA complex and binding of dissociated Gd ions is restarted.

Without being limited by theoretical considerations, placing the selective exchange medium in direct contact with a dry macrocyclic chelated GBCA complex will not activate the selective exchange medium. Instead, the addition of water is expected to activate the selective exchange medium. In this context, activation refers to the process of inducing the selective exchange medium to remove Gd ions from partially coordinated complexes of the macrocyclic chelated GBCA and bind the dissociated Gd ions.

In certain embodiments, the present disclosure provides a dry composition comprising a macrocyclic chelated GBCA and a selective exchange medium, wherein both the GBCA and the selective exchange medium are in a dry state. In such embodiments, the selective exchange medium remains substantially inactive while dry and is configured to become active only upon hydration, such that upon addition of water the selective exchange medium induces dechelation of Gd ions from partially coordinated complexes and binds dissociated Gd ions.

A third set of example methods according to the disclosure is referred to as drying methods. In these methods and the other methods of the present disclosure, when water is removed, the removed water is referred to as free water, which is water that is not coordinated to the GBCA complexes. After drying, the macrocyclic chelated GBCA complexes can be reconstituted immediately prior to administration. Upon reconstitution, a selective exchange medium can be activated by contact with the macrocyclic chelated GBCA complex in solution.

As used herein, “bound water” refers to water molecules that remain associated with a macrocyclic gadolinium-based contrast agent in a dried solid state after removal of unbound or free water, including water that is retained through coordination, hydration, or other non-free associations with the gadolinium complex. Bound water is distinguished from free or unbound water, which can be removed during conventional drying processes.

Bound water content of dry GBCA compositions can be determined using analytical techniques suitable for measuring residual water in solid materials, including thermogravimetric analysis (TGA), Karl Fischer titration, differential scanning calorimetry (DSC), or combinations thereof. Such techniques enable quantification of bound water remaining in the dried composition and confirmation that the bound water content is less than 5 wt %, 4 wt %, 3 wt %, 2 wt %, 1 wt %, or 0.5 wt %.

In certain embodiments, the dried macrocyclic chelated GBCA is stored and provided without any selective exchange medium. In such embodiments, the dry GBCA composition is free of exchange resins, membranes, or other selective exchange media, and stability is achieved primarily through reduction of free water content while maintaining the coordinated inner-sphere water molecule.

In one embodiment, a macrocyclic chelated GBCA complex is processed immediately after a final exchange media treatment and filtration to remove water from the product within a controlled timeframe. The water removal is conducted in less than 24 hours, preferably less than 8 hours, and more preferably in less than 3 hours.

A batch of the macrocyclic chelated GBCA complex can be maintained either with the selective exchange medium present or at just above the freezing point after selective exchange medium isolation, while preparing for subsequent processing.

The macrocyclic chelated GBCA complex is lyophilized to remove water. The freeze temperature can be maintained at −30 °C and −45 °C, and sublimation is conducted under extremely low vacuum conditions to facilitate sublimation at the lowest possible temperature. The process can be conducted in partially filled and partially capped vials, such as 10 mL of product in a 50 mL vial. Thereafter, the macrocyclic chelated GBCA complex can be reconstituted immediately prior to administration to a patient.

Alternatively, continuous processing can be employed by removing the selective exchange medium via inline filtration and feeding the filtrate to an atomizer, which discharges into a stream of heated nitrogen (spray drying). The water is flash evaporated, and the macrocyclic chelated GBCA complex, containing 1% to 5% residual moisture, is collected and packaged. Thereafter, the macrocyclic chelated GBCA complex can be reconstituted immediately prior to administration to a patient.

A lyophilization process includes maintaining the macrocyclic chelated GBCA complex at 3 °C to 8 °C above freezing after a further or final exchange media treatment, freezing the macrocyclic chelated GBCA complex within 8 hours, preferably within 2 hours, at −40 °C or lower, sublimating water down to 3 to 5% residual moisture at a primary drying temperature of −37 C to −35 C. Thereafter, the macrocyclic chelated GBCA complex can be reconstituted immediately prior to administration to a patient.

A flash evaporation method includes feeding filtrate macrocyclic chelated GBCA complex from exchange media treatment into a flash evaporator, atomizing into a heated nitrogen stream (175 °C to 185 °C) or alternatively, vacuum spray drying at 40 °C to 60 °C. Solid product is collected and packaged under dry nitrogen or low-humidity air. Thereafter, the macrocyclic chelated GBCA complex can be reconstituted immediately prior to administration to a patient.

It is contemplated that spray drying can be carried out in either of two general gas-handling configurations. The first is a closed recirculating nitrogen-purged system, in which nitrogen is used as an atomization gas, and the system is operated under nitrogen purge with recirculation. The second is an open single-pass hot nitrogen system with hot nitrogen in and exhaust out, optionally operated under reduced pressure (e.g., vacuum spray drying).

Other water removal methods are contemplated. For example, these include concentration via evaporation and cooling to crystallize the product, followed by drying and solvent crystallization and drying.

Drying can include primary and secondary drying, making it a two-stage process.

After the drying is performed and prior to administering to a patient, the macrocyclic chelated GBCA complex can be reconstituted into a solution and contacted with a selective exchange medium. Contact with the selective exchange medium activates the medium so that exchange media treatment occurs.

A fourth set of example methods according to the disclosure is referred to as combination methods involving mix-on-demand and drying or freezing.

Each of these methods improves processing, storage, and administration techniques for macrocyclic GBCAs, ensuring chelate integrity, reducing partially coordinated complexes, and enhancing shelf life.

An exemplary synthesis and exchange media treatment of a macrocyclic chelated GBCA complex, namely Gd-DOTA, will now be described.

One mole of DOTA is added to 1 L of distilled water in a vessel under mechanical stirring. The reaction temperature is raised to 90 to 98 °C. When the temperature is reached, gadolinium oxide is added to the reaction volume incrementally over a period of, for example, 30 minutes. Gadolinium oxide additions are made when the reaction solution turns clear, or the amount of free DOTA reaches 0.005 vol % to 0.05 vol % on a final product basis, and free gadolinium ion is preferably less than 10 ppm mol-wt, more preferably less than 1.5 ppm mol-wt, and most preferably less than 1.0 ppm mol-wt on a final product basis. The reaction mixture is conducted at 95 °C for at least 24 hours, preferably 32 hours, and more preferably 40 hours, including any and all subranges therebetween. Post complexation, the solution is cooled to 35 °C to 45 °C, and meglumine is introduced to neutralize the acidic medium, adjusting the pH to 7.0 to 7.4.

Exchange media treatment can be performed using a selective exchange medium, preferably a trivalent-cation selective exchange medium

By way of non-limiting example, a strong-acid cationic exchange resin with sulfonic acid functional groups (e.g., 50W-X8 or X12 conditioned with MegH+) is selected for its high capacity (2.0 meq/mL) and selectivity for trivalent charge cations. A particle size of 100-400 mesh can be chosen to increase the rate of reaction.

The resin can be pretreated by sequential washing with an acid to protonate functional groups, deionized water to neutralize pH, and meglumine solution to condition the resin for counterion exchange.

The present disclosure has found that such a synthesis and exchange media treatment, for example with a resin, can achieve a product with about 99.5% fully coordinated complexes, it is only achieved for a limited period, typically 12 to 48 hours, due to the tendency to shift back to an equilibrium concentration typically that settles at 97 to 99% fully coordinated complexes and 1 to 3% partially coordinated complexes.

The selective exchange medium according to the present disclosure can be configured to induce dechelation of Gd ions from partially coordinated complexes of the macrocyclic chelated GBCA complex and bind dissociated Gd ions. The resin form of a selective exchange medium should be applied to the macrocyclic chelated GBCA complex at 0.2 to 2.5 molar equivalents, preferably 0.1 to 2.0, 0.3 to 1.5, 0.5 to 2.0, 0.5 to 1.5, 0.9 to 1.5, and more preferably 1.0.

One or more exchange media treatments are also contemplated. They can be at the same molar equivalence or different. For example, when there are two exchange media treatments, a first exchange media treatment can be at 1.0 molar equivalents, while the second exchange media treatment can be at 0.3 molar equivalents. The order of exchange media treatments can be at decreasing equivalents, increasing equivalents, or constant equivalents. When there are three or more exchange media treatments, the equivalents used can be combinations of the foregoing.

Non-limiting examples of suitable selective exchange media will now be described.

Cationic exchange resins are particularly effective for this purpose due to their ability to selectively bind and remove positive gadolinium ions from aqueous solutions. Among the suitable resins, strong-acid cation exchangers like those based on styrene-divinylbenzene (DVB) copolymers are preferred. These exchange resins typically feature sulfonic acid functional groups (—SO₃H), which provide high affinity for trivalent cations, including, particularly, gadolinium.

Strong-acid cation resins such as Seplife™ 50Wx8s and 50Wx4s, Ag® 50W-X8/12, H+ and Purolite® C150, H+ are particularly suitable once converted to the meglumine H+ counterion form.

Seplife™ 50Wx8s and 50Wx4s are commercially available from Sunresin New Materials Co., Ltd. They are polymeric bead materials distinguished primarily by their cross-linking degree (8% vs. 4% divinylbenzene content in the polymer matrix), which imparts distinct physical and functional characteristics

Ag® 50W and Purolite® C150 are polystyrene strong-acid resin crosslinked with divinylbenzene. The functional group is sulfonic acid. Purolite® C150 is macroporous, and Ag® 50W is microporous. An ultrapure equivalent of Purolite® C150 is UltraClean™ UCW9126, H+.

Ag® 50W is commercially available as hydrogen-form resin from Bio-Rad Laboratories Inc. Purolite® C150 and UltraClean™ UCW9126 are commercially available from Purolite Company in the H+ and Na+ forms.

Selective exchange media that are exchange resins can also be a resin selected from the group consisting of: iminodiacetic acid, ethylenediaminetetraacetic acid, and diethylenetriaminepentaacetic acid, thiourea, strong-acid cation, weak acid cation, and chelating resins. Other exchange resins are contemplated, including mixed-mode cation exchange and mixed-mode anion-exchange.

A Sartobind® S membrane filter (Sartorius AG, Gottingen, Germany) is also suitable as the selective exchange medium. The Sartobind S membrane is a strong cation exchange medium incorporating sulfonic acid functional groups immobilized on a porous membrane substrate. When used in the present methods and systems, the membrane provides rapid, uniform ion exchange due to its high surface area and convective flow characteristics. The membrane can selectively bind trivalent cations such as Gd ions that have dissociated from partially coordinated GBCA complexes, thereby decreasing the fraction of partially coordinated complexes and increasing the proportion of fully coordinated complexes in the treated solution. The membrane can be employed as a discrete insert, cartridge, or inline element within a syringe, filter assembly, microchannel device, or power injector line, as shown in FIGS. 1 to 6, and can be combined with other ion exchange media or resins to achieve the desired capacity and flow performance.

Other membrane-based materials can also serve as selective exchange medium 130. Suitable alternatives include carboxymethyl-functionalized membranes, iminodiacetate-functionalized membranes, phosphonate-functionalized membranes, sulfonate-functionalized membranes, and mixed-functionality membranes incorporating combinations thereof.

Time course experiments were conducted to evaluate the percentage of fully and partially coordinated complexes before and after exchange media treatment. The test procedure described herein is the Resin-UV Comparative Protocol.

To a 20 mL screw-capped vial were added 1.0 g of washed Purolite® C150 ion exchange resin, 5.0 g of deionized water, and a magnetic stir bar. The mixture was stirred at ambient temperature for about 24 h. A gadoterate meglumine solution (approx. 0.5 mmol/mL) (5.0 g) was added to the vial as quickly as possible. The end of the addition was taken as t=0. The resulting solution was stirred at ambient temperature. Samples for UV analysis were collected at suitable time points, with the first at t=1 minute. The samples were filtered with a 0.45 μm syringe filter directly into a cuvette for UV data acquisition, then returned to the vial.

Depending on the number of sampling timepoints, the volume required for UV data acquisition, and the quantity of the Purolite® C150 resin/water/gadoterate meglumine mixture, one might choose not to return the filtered sample. If so desired, one can use an aqueous salt solution, such as, but not limited to, sodium chloride, instead of the deionized water to modify the swelling state of the resin. The UV spectra for the time points were acquired over the range of 250 to 350 nm.

The absorbance for the peak at 274 nm was corrected for changes in baseline by linearly interpolating the baseline from before and after the group of peaks around 274 nm (i.e., approximately from 270 to 280 nm) and subtracting the interpolated baseline value at 274 nm from the absorbance of the 274 nm peak maximum.

In the Resin-UV Comparative Protocol, for the analysis of a timeseries experiment, it is useful to plot the corrected absorbances of the 274 nm peak as a percentage of the corrected absorbances of the 274 nm peak at the first timepoint versus time. The decline from 100% at the first time point over the course of the experiment to the end value can be interpreted as the reduction in partially coordinated gadolinium complexes.

A simple way of comparing the content of partially coordinated gadolinium complexes between compounds is then to compare the percentage values at the last time point. In an alternate way, compounds can be compared by designating one compound as the reference and dividing the corrected absorbances of the 274 nm peak at each time point by the corresponding absorbances of the 274 nm peak of the reference. When these ratios are plotted versus time, the reference compound shows a straight line at 100% and the other compounds are above that line if they contain fewer partially coordinated complexes and below that line if they contain more partially coordinated complexes.

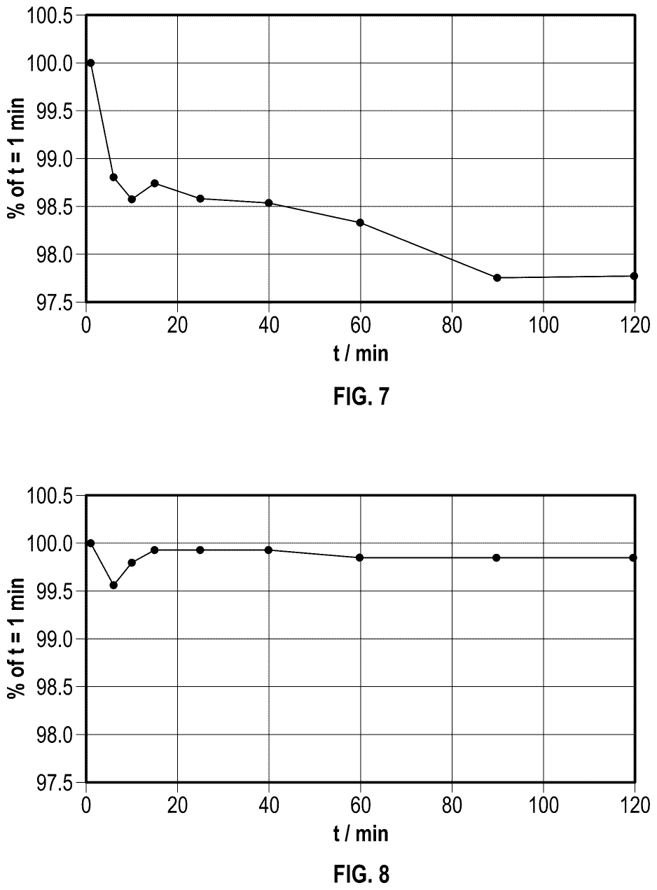

Reference is made to FIG. 7 showing a timeseries testing for fully and partially coordinated complexes of commercially available Gd-DOTA sold under the trade name Dotarem® (Guerbet LLC). The X axis is the gadoterate concentration at each time point, divided by t=1 minute gadoterate concentration expressed in percent.

As can be seen, the composition has approximately 97.8% fully coordinated complexes and 2.2% partially coordinated complexes. Approximately 22,000 ppm Gd ions were released during the 120-minute test. The data is shown in Table 1A.

TABLE 1A
Dotarem ®

| | | Peak | | | | | |
|---|---|---|---|---|---|---|---|
| | | height | | | Y axis | Peak | % of |
| | Time | (274 | | | scale | height | AS14- |
| | post | nm) / | Y | Y axis / | factor / | (274 | 42- |
| Sample | mix/min | cm | axis/cm | A | A/cm | nm) / A | 22(1) |
| AS14-42-22(1) | 1 | 22.10 | 23.55 | 0.50 | 0.02123 | 0.4692 | 100.0 |
| AS14-42-22(2) | 6 | 22.15 | 21.50 | 0.45 | 0.02093 | 0.4636 | 98.8 |
| AS14-42-22(3) | 10 | 22.15 | 21.55 | 0.45 | 0.02088 | 0.4625 | 98.6 |
| AS14-42-22(4) | 15 | 22.10 | 23.85 | 0.50 | 0.02096 | 0.4633 | 98.7 |
| AS14-42-22(5) | 25 | 22.10 | 21.50 | 0.45 | 0.02093 | 0.4626 | 98.6 |
| AS14-42-22(6) | 40 | 22.10 | 23.90 | 0.50 | 0.02092 | 0.4623 | 98.5 |
| AS14-42-22(7) | 60 | 22.10 | 23.95 | 0.50 | 0.02088 | 0.4614 | 98.3 |
| AS14-42-22(8) | 90 | 22.20 | 24.20 | 0.50 | 0.02066 | 0.4587 | 97.8 |
| AS14-42-22(9) | 120 | 22.25 | 24.25 | 0.50 | 0.02062 | 0.4588 | 97.8 |

Reference is made to FIG. 8 showing a timeseries testing for fully and partially coordinated complexes of commercially available Gd-DOTA sold under the trade name Dotarem® (Guerbet LLC) that, prior to testing, has been treated with Bio-Rad Laboratories Inc. Ag® 50W-X8 conditioned with MegH+ resin at 1.0 molar equivalent for 2 hours. The X axis is the gadoterate concentration at each time point divided by the initial gadoterate concentration expressed in percent.

As can be seen, the composition has approximately 99.8% fully coordinated complexes and 0.2% partially coordinated complexes. The data is shown in Table 1B.

After exchange media treatment with the resin, the number of partially coordinated complexes has been reduced by 75 to 90%, that is, only 2,000-5,000 ppm of Gd ions were released during the 120-minute test.

It should be noted, however, that what appears in this data set to be high-purity is temporary, and after several days, if the test is repeated on the batch, the data will resemble that of Table 1A since the equilibrium of fully coordinated vs. partially coordinated complexes will shift back. However, if, according to the present disclosure's mix-on-demand methods, the exchange media treatment is performed just prior to administering to a patient, then a product with 99.8% fully coordinated complexes and 0.2% partially coordinated complexes can be administered, greatly enhancing the safety to the patient.

TABLE 1B
Dotarem ® With Exchange Media Treatment.

| | | Peak | | | | | |
|---|---|---|---|---|---|---|---|
| | | height | | | Y axis | Peak | % of |
| | Time | (274 | | | scale | height | AS14- |
| | post | nm) / | Y | Y axis / | factor / | (274 | 42- |
| Sample | mix/min | cm | axis/cm | A | A/cm | nm) / A | 24(1) |
| AS14-42-24(1) | 1 | 21.00 | 22.60 | 0.50 | 0.02212 | 0.4646 | 100.0 |
| AS14-42-24(2) | 6 | 21.00 | 22.70 | 0.50 | 0.02203 | 0.4626 | 99.6 |
| AS14-42-24(3) | 10 | 21.05 | 22.70 | 0.50 | 0.02203 | 0.4637 | 99.8 |
| AS14-42-24(4) | 15 | 21.15 | 20.50 | 0.45 | 0.02195 | 0.4643 | 99.9 |
| AS14-42-24(5) | 25 | 21.15 | 20.50 | 0.45 | 0.02195 | 0.4643 | 99.9 |
| AS14-42-24(6) | 40 | 21.15 | 20.50 | 0.45 | 0.02195 | 0.4643 | 99.9 |
| AS14-42-24(7) | 60 | 21.20 | 22.85 | 0.50 | 0.02188 | 0.4639 | 99.8 |
| AS14-42-24(8) | 90 | 21.20 | 22.85 | 0.50 | 0.02188 | 0.4639 | 99.8 |
| AS14-42-24(9) | 120 | 21.20 | 22.85 | 0.50 | 0.02188 | 0.4639 | 99.8 |

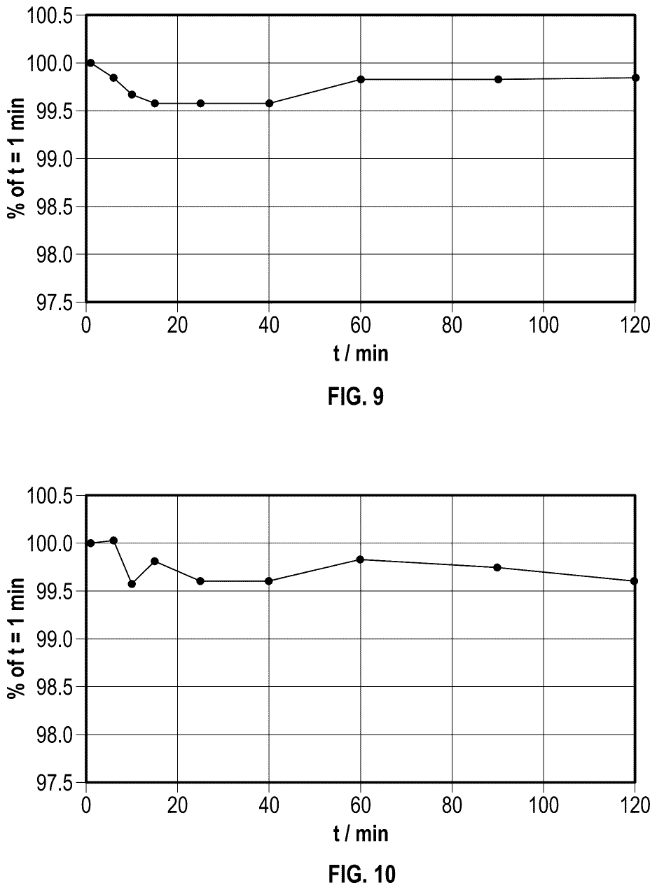

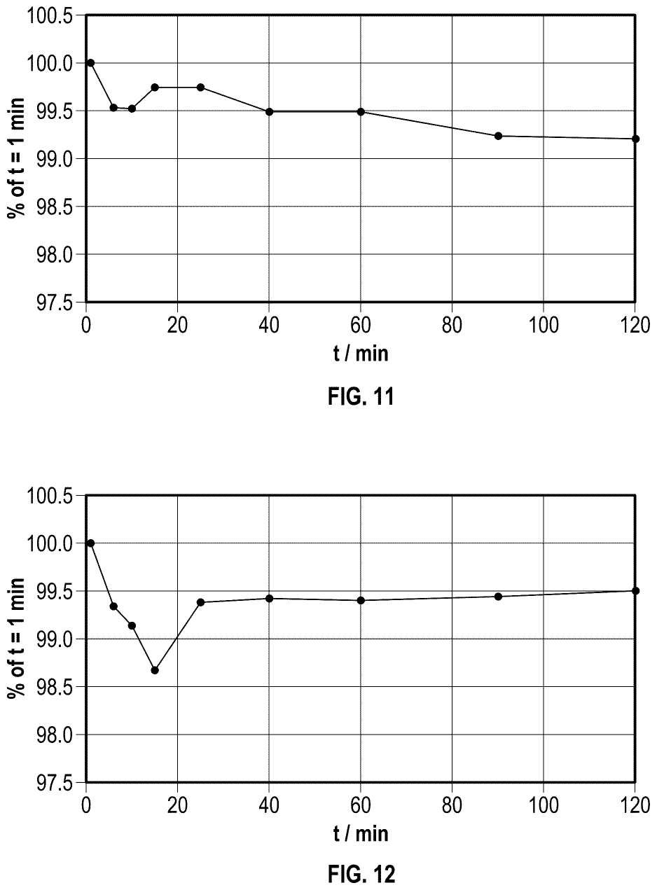

Reference is made to FIGS. 9, 10, and 11 that show a timeseries testing for fully and partially coordinated complexes of Gd-DOTA synthesized according to the exemplary synthesis described above, treated with Ag® 50W-X8 conditioned with MegH+ resin at 1.0 molar equivalent for 2 hours. FIGS. 9, 10, and 11 show three different time points that have elapsed after the resin has been removed, namely 2h45 min, 5h15 min, and 26h20 min, respectively. The data for FIGS. 9, 10, and 11 are shown in Tables 2A, 2B, and 2C, respectively.

As can be seen, over time, the composition degrades from approximately 99.8% fully coordinated complexes and 0.2% partially coordinated complexes to 99.2% fully coordinated complexes and 0.8% partially coordinated complexes. This occurs over the course of less than 2 days. It is expected that if several days pass and the test is repeated, the data should resemble that of FIG. 7 and Table 1A since the equilibrium will shift back. However, with the methods of the present disclosure, the equilibrium shift can be halted or delayed.

TABLE 2A
Gd-DOTA At 2 h 45 min Post Exchange Media Treatment

| | | Peak | | | | | |
|---|---|---|---|---|---|---|---|
| | | height | | | Y axis | Peak | |
| | Time | (274 | | Y | scale | height | % of |
| | post | nm) / | Y | axis / | factor / | (274 nm) / | AS15- |
| Sample | mix/min | cm | axis/cm | A | A/cm | A | 62-5(1) |
| AS15-62-5(1) | 1 | 21.25 | 22.05 | 0.50 | 0.02268 | 0.4819 | 100.0 |
| AS15-62-5(2) | 6 | 21.30 | 24.35 | 0.55 | 0.02259 | 0.4811 | 99.8 |
| AS15-62-5(3) | 10 | 21.35 | 24.45 | 0.55 | 0.02249 | 0.4803 | 99.7 |
| AS15-62-5(4) | 15 | 21.40 | 22.30 | 0.50 | 0.02242 | 0.4798 | 99.6 |
| AS15-62-5(5) | 25 | 21.40 | 22.30 | 0.50 | 0.02242 | 0.4798 | 99.6 |
| AS15-62-5(6) | 40 | 21.40 | 22.30 | 0.50 | 0.02242 | 0.4798 | 99.6 |
| AS15-62-5(7) | 60 | 21.55 | 22.40 | 0.50 | 0.02232 | 0.4810 | 99.8 |
| AS15-62-5(8) | 90 | 21.55 | 22.40 | 0.50 | 0.02232 | 0.4810 | 99.8 |
| AS15-62-5(9) | 120 | 21.65 | 22.50 | 0.50 | 0.02222 | 0.4811 | 99.8 |

TABLE 2B
Gd-DOTA At 5 h 15 min Post Exchange Media Treatment

| | | Peak | | | Y axis | Peak | |
|---|---|---|---|---|---|---|---|
| | Time | height | | | scale | height | % of |
| | post | (274 | Y | Y axis / | factor / | (274 | AS15- |
| Sample | mix/min | nm) / cm | axis/cm | A | A/cm | nm) / A | 62-6(1) |
| AS15-62-6(1) | 1 | 20.95 | 21.80 | 0.50 | 0.02294 | 0.4805 | 100.0 |
| AS15-62-6(2) | 6 | 21.10 | 21.95 | 0.50 | 0.02278 | 0.4806 | 100.0 |
| AS15-62-6(3) | 10 | 21.10 | 22.05 | 0.50 | 0.02268 | 0.4785 | 99.6 |
| AS15-62-6(4) | 15 | 21.15 | 22.05 | 0.50 | 0.02268 | 0.4796 | 99.8 |
| AS15-62-6(5) | 25 | 21.25 | 22.20 | 0.50 | 0.02252 | 0.4786 | 99.6 |
| AS15-62-6(6) | 40 | 21.25 | 22.20 | 0.50 | 0.02252 | 0.4786 | 99.6 |
| AS15-62-6(7) | 60 | 21.25 | 22.15 | 0.50 | 0.02257 | 0.4797 | 99.8 |
| AS15-62-6(8) | 90 | 21.35 | 24.50 | 0.55 | 0.02245 | 0.4793 | 99.7 |
| AS15-62-6(9) | 120 | 21.45 | 24.65 | 0.55 | 0.02231 | 0.4786 | 99.6 |

TABLE 2C
Gd-DOTA At 26 h 20 min Post Exchange Media Treatment

| | | Peak | | | | | |
|---|---|---|---|---|---|---|---|
| | | height | | | Y axis | Peak | |
| | Time | (274 | | Y | scale | height | % of |
| | post | nm) / | Y | axis / | factor / | (274 nm) / | AS15- |
| Sample | mix/min | cm | axis/cm | A | A/cm | A | 62-7(1) |
| AS15-62-7(1) | 1 | 21.25 | 22.45 | 0.50 | 0.02227 | 0.4733 | 100.0 |
| AS15-62-7(2) | 6 | 21.25 | 20.30 | 0.45 | 0.02217 | 0.4711 | 99.5 |
| AS15-62-7(3) | 10 | 21.30 | 20.35 | 0.45 | 0.02211 | 0.4710 | 99.5 |
| AS15-62-7(4) | 15 | 21.40 | 20.40 | 0.45 | 0.02206 | 0.4721 | 99.7 |
| AS15-62-7(5) | 25 | 21.40 | 20.40 | 0.45 | 0.02206 | 0.4721 | 99.7 |
| AS15-62-7(6) | 40 | 21.45 | 20.50 | 0.45 | 0.02195 | 0.4709 | 99.5 |
| AS15-62-7(7) | 60 | 21.45 | 20.50 | 0.45 | 0.02195 | 0.4709 | 99.5 |
| AS15-62-7(8) | 90 | 21.50 | 20.60 | 0.45 | 0.02184 | 0.4697 | 99.2 |
| AS15-62-7(9) | 120 | 21.65 | 20.75 | 0.45 | 0.02169 | 0.4695 | 99.2 |

Reference is made to FIG. 12, which shows a timeseries testing for fully and partially coordinated complexes of Gd-DOTA synthesized according to the exemplary synthesis described above, treated with Ag® 50W-X8 conditioned with MegH+ resin at 1.0 molar equivalent for 2 hours, and then frozen for 17.5 days prior to the timeseries testing.

For this experiment, a 20 mL vial containing 11.5 g of gadoterate meglumine was removed from the freezer at −49 °C and allowed to thaw at ambient temperature for 10 minutes in air, then in a water bath at ambient temperature. The vial contents were completely melted after 17 minutes and at 16 °C. A pH probe was inserted, and about 45 minutes after taking the vial out of the freezer, the pH was steady at 7.13, i.e., there were no changes due to temperature changes, and the ambient temperature had been reached. The time course experiment was started 50 minutes after taking the vial out of the freezer. The data is shown in Table 3.

It can be seen that at t=120 minutes, fully coordinated complexes are at 99.5% and partially coordinated complexes are at 0.5% of the composition. Again, this had been held for approximately 420 hours

TABLE 3
Gd-DOTA Post Exchange Media Treatment After 14 Days Freezing

| | | Peak | | | | | |
|---|---|---|---|---|---|---|---|
| | | height | | | Y axis | Peak | |
| | Time | (274 | | Y | scale | height | % of |
| | post | nm) / | Y | axis / | factor / | (274 nm) / | AS15- |
| Sample | mix/min | cm | axis/cm | A | A/cm | A | 62-9(1) |
| AS15-62-9(1) | 1 | 20.20 | 21.90 | 0.45 | 0.02055 | 0.4151 | 100.0 |
| AS15-62-9(2) | 6 | 20.25 | 22.10 | 0.45 | 0.02036 | 0.4123 | 99.3 |
| AS15-62-9(3) | 10 | 20.30 | 22.20 | 0.45 | 0.02027 | 0.4115 | 99.1 |
| AS15-62-9(4) | 15 | 20.25 | 22.25 | 0.45 | 0.02022 | 0.4096 | 98.7 |
| AS15-62-9(5) | 25 | 20.35 | 22.20 | 0.45 | 0.02027 | 0.4125 | 99.4 |
| AS15-62-9(6) | 40 | 20.45 | 22.30 | 0.45 | 0.02018 | 0.4127 | 99.4 |
| AS15-62-9(7) | 60 | 20.40 | 22.25 | 0.45 | 0.02022 | 0.4126 | 99.4 |
| AS15-62-9(8) | 90 | 20.50 | 22.35 | 0.45 | 0.02013 | 0.4128 | 99.4 |
| AS15-62-9(9) | 120 | 20.65 | 22.50 | 0.45 | 0.02000 | 0.4130 | 99.5 |

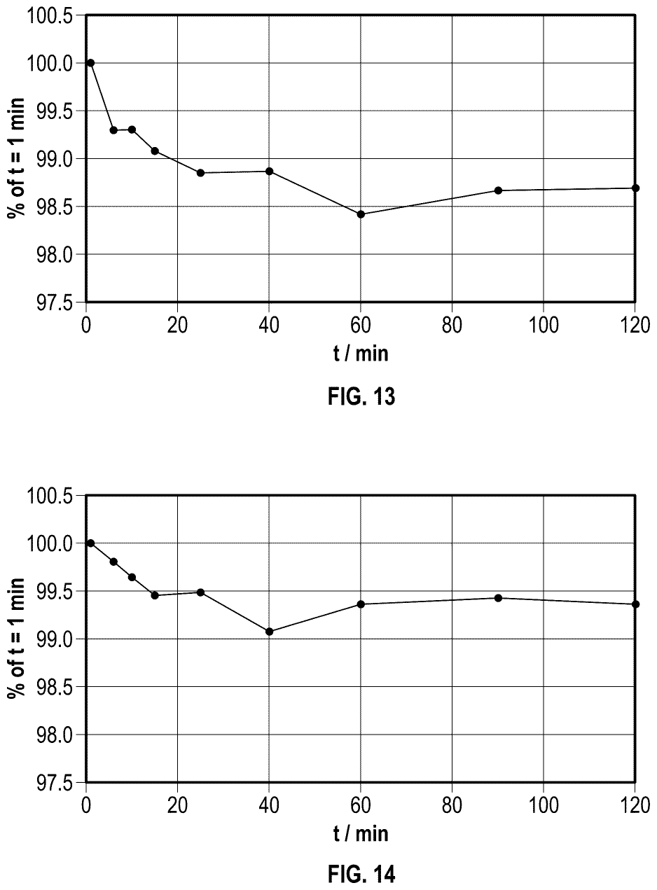

Reference is made to FIG. 13 that shows a timeseries testing for fully and partially coordinated complexes of Gd-DOTA synthesized according to the exemplary synthesis described above, treated with Ag® 50W-X8 conditioned with MegH+ resin for 2 hours and then frozen for 56 days, then stirred at ambient temperature for 6 days with 0.3 eq. of resin prior to the timeseries testing. The data is shown in Table 4.

It can be seen that at t=120 minutes, fully coordinated complexes are at 98.7%, and partially coordinated complexes are at 1.3% of the composition.

TABLE 4
Gd-DOTA Post Exchange Media Treatment, Frozen With 0.3 Eq. Resin
For 56 Days, Thawed, And Mixed For 6 Days At Ambient Temp

| | | Peak | | | | | |
|---|---|---|---|---|---|---|---|
| | | height | | | Y axis | Peak | % of |
| | Time | (274 | | Y | scale | height | AS15- |
| | post | nm) / | Y | axis / | factor / | (274 | 62- |
| Sample | mix/min | cm | axis/cm | A | A/cm | nm) / A | 19(1) |
| AS15-62-19(1) | 1 | 19.85 | 20.30 | 0.45 | 0.02217 | 0.4400 | 100.0 |
| AS15-62-19(2) | 6 | 20.05 | 20.65 | 0.45 | 0.02179 | 0.4369 | 99.3 |
| AS15-62-19(3) | 10 | 20.10 | 20.70 | 0.45 | 0.02174 | 0.4370 | 99.3 |
| AS15-62-19(4) | 15 | 20.20 | 20.85 | 0.45 | 0.02158 | 0.4360 | 99.1 |
| AS15-62-19(5) | 25 | 20.25 | 20.95 | 0.45 | 0.02148 | 0.4350 | 98.8 |
| AS15-62-19(6) | 40 | 20.35 | 21.05 | 0.45 | 0.02138 | 0.4350 | 98.9 |
| AS15-62-19(7) | 60 | 20.45 | 21.25 | 0.45 | 0.02118 | 0.4331 | 98.4 |
| AS15-62-19(8) | 90 | 20.55 | 21.30 | 0.45 | 0.02113 | 0.4342 | 98.7 |
| AS15-62-19(9) | 120 | 20.70 | 21.45 | 0.45 | 0.02098 | 0.4343 | 98.7 |

Reference is made to FIG. 14 that shows a timeseries testing for fully and partially coordinated complexes of Gd-DOTA synthesized according to the exemplary synthesis described above, treated with Ag® 50W-X8 conditioned with MegH+ resin for 2 hours, and then frozen for 63 days, then stirred at ambient temperature for 1.5 hours with 0.3 eq. of resin prior to the timeseries testing. The data is shown in Table 5.

It can be seen that at t=120 minutes, fully coordinated complexes are at 99.4%, and partially coordinated complexes are at 0.6% of the composition.

TABLE 5
Gd-DOTA Post Exchange Media Treatment, Frozen 63 Days,
Thawed, And Mixed For 2 Hrs. At Ambient Temp

| | Time | Peak | | | Y axis | Peak | % of |
|---|---|---|---|---|---|---|---|
| | post | height | Y | | scale | height | AS15- |
| | mix/ | (274 | axis/ | Y | factor/ | (274 | 62- |
| Sample | min | nm)/cm | cm | axis/A | A/cm | nm)/A | 20(1) |
| AS15-62- | 1 | 19.25 | 21.95 | 0.50 | 0.02278 | 0.4385 | 100.0 |
| 20(1) | | | | | | | |
| AS15-62- | 6 | 19.30 | 22.05 | 0.50 | 0.02268 | 0.4376 | 99.8 |
| 20(2) | | | | | | | |
| AS15-62- | 10 | 19.40 | 22.20 | 0.50 | 0.02252 | 0.4369 | 99.6 |
| 20(3) | | | | | | | |
| AS15-62- | 15 | 19.45 | 22.30 | 0.50 | 0.02242 | 0.4361 | 99.5 |
| 20(4) | | | | | | | |
| AS15-62- | 25 | 19.50 | 22.35 | 0.50 | 0.02237 | 0.4362 | 99.5 |
| 20(5) | | | | | | | |
| AS15-62- | 40 | 19.55 | 22.50 | 0.50 | 0.02222 | 0.4344 | 99.1 |
| 20(6) | | | | | | | |
| AS15-62- | 60 | 19.65 | 22.55 | 0.50 | 0.02217 | 0.4357 | 99.4 |
| 20(7) | | | | | | | |
| AS15-62- | 90 | 19.75 | 22.65 | 0.50 | 0.02208 | 0.4360 | 99.4 |
| 20(8) | | | | | | | |
| AS15-62- | 120 | 19.80 | 20.45 | 0.45 | 0.02200 | 0.4357 | 99.4 |
| 20(9) | | | | | | | |

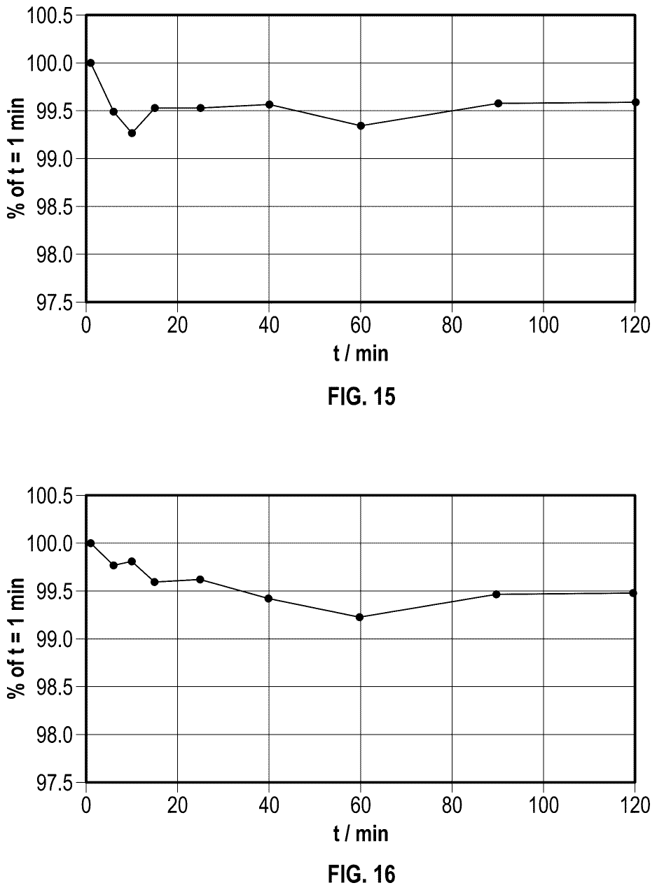

Reference is made to FIG. 15 that shows a timeseries testing for fully and partially coordinated complexes of Gd-DOTA synthesized according to the exemplary synthesis described above, treated with Ag® 0W-X8 conditioned with MegH+ resin for 2 hours, and then frozen for 69 days, then stirred at ambient temperature for 1 hour with the resin prior to the timeseries testing. The data is shown in Table 6.

It can be seen that at t=120 minutes, fully coordinated complexes are at 99.6%, and partially coordinated complexes are at 0.4% of the composition.

TABLE 6
Gd-DOTA Post Exchange Media Treatment, Frozen For 69
Days, Thawed, And Mixed For 1 Hr. At Ambient Temp

| | Time | Peak | | | Y axis | Peak | % of |
|---|---|---|---|---|---|---|---|
| | post | height | Y | | scale | height | AS15- |
| | mix/ | (274 | axis/ | Y | factor/ | (274 | 62- |
| Sample | min | nm)/cm | cm | axis/A | A/cm | nm)/A | 21(1) |
| AS15-62- | 1 | 19.70 | 22.70 | 0.55 | 0.02423 | 0.4773 | 100.0 |
| 21(1) | | | | | | | |
| AS15-62- | 6 | 19.85 | 20.90 | 0.50 | 0.02392 | 0.4749 | 99.5 |
| 21(2) | | | | | | | |
| AS15-62- | 10 | 19.90 | 21.00 | 0.50 | 0.02381 | 0.4738 | 99.3 |
| 21(3) | | | | | | | |
| AS15-62- | 15 | 20.00 | 21.05 | 0.50 | 0.02375 | 0.4751 | 99.5 |
| 21(4) | | | | | | | |
| AS15-62- | 25 | 20.00 | 21.05 | 0.50 | 0.02375 | 0.4751 | 99.5 |
| 21(5) | | | | | | | |
| AS15-62- | 40 | 20.15 | 21.20 | 0.50 | 0.02358 | 0.4752 | 99.6 |
| 21(6) | | | | | | | |
| AS15-62- | 60 | 20.20 | 21.30 | 0.50 | 0.02347 | 0.4742 | 99.3 |
| 21(7) | | | | | | | |
| AS15-62- | 90 | 20.20 | 21.25 | 0.50 | 0.02353 | 0.4753 | 99.6 |
| 21(8) | | | | | | | |
| AS15-62- | 120 | 20.25 | 21.30 | 0.50 | 0.02347 | 0.4754 | 99.6 |
| 21(9) | | | | | | | |

Reference is made to FIG. 16 that shows a timeseries testing for fully and partially coordinated complexes of Gd-DOTA synthesized according to the exemplary synthesis described above, treated with Ag® 50W-X8 conditioned with MegH+ resin at 1.0 molar equivalents for 2 hours, and then frozen for 70 days, then stirred at ambient temperature for 4 hours with the resin prior to the timeseries testing. The data is shown in Table 7.

It can be seen that at t=120 minutes, fully coordinated complexes are at 99.5%, and partially coordinated complexes are at 0.5% of the composition.

TABLE 7
Gd-DOTA Post Exchange Media Treatment, Frozen For 70
Days, Thawed, And Mixed For 4 Hrs. At Ambient Temp

| | Time | Peak | | | Y axis | Peak | % of |
|---|---|---|---|---|---|---|---|
| | post | height | Y | | scale | height | AS15- |
| | mix/ | (274 | axis/ | Y | factor/ | (274 | 62- |
| Sample | min | nm)/cm | cm | axis/A | A/cm | nm)/A | 22(1) |
| AS15-62- | 1 | 20.35 | 21.50 | 0.50 | 0.02326 | 0.4733 | 100.0 |
| 22(1) | | | | | | | |
| AS15-62- | 6 | 20.35 | 21.55 | 0.50 | 0.02320 | 0.4722 | 99.8 |
| 22(2) | | | | | | | |
| AS15-62- | 10 | 20.50 | 21.70 | 0.50 | 0.02304 | 0.4724 | 99.8 |
| 22(3) | | | | | | | |
| AS15-62- | 15 | 20.55 | 21.80 | 0.50 | 0.02294 | 0.4713 | 99.6 |
| 22(4) | | | | | | | |
| AS15-62- | 25 | 20.65 | 21.90 | 0.50 | 0.02283 | 0.4715 | 99.6 |
| 22(5) | | | | | | | |
| AS15-62- | 40 | 20.75 | 22.05 | 0.50 | 0.02268 | 0.4705 | 99.4 |
| 22(6) | | | | | | | |
| AS15-62- | 60 | 20.85 | 22.20 | 0.50 | 0.02252 | 0.4696 | 99.2 |
| 22(7) | | | | | | | |
| AS15-62- | 90 | 20.90 | 22.20 | 0.50 | 0.02252 | 0.4707 | 99.5 |
| 22(8) | | | | | | | |
| AS15-62- | 120 | 20.95 | 22.25 | 0.50 | 0.02247 | 0.4708 | 99.5 |
| 22(9) | | | | | | | |

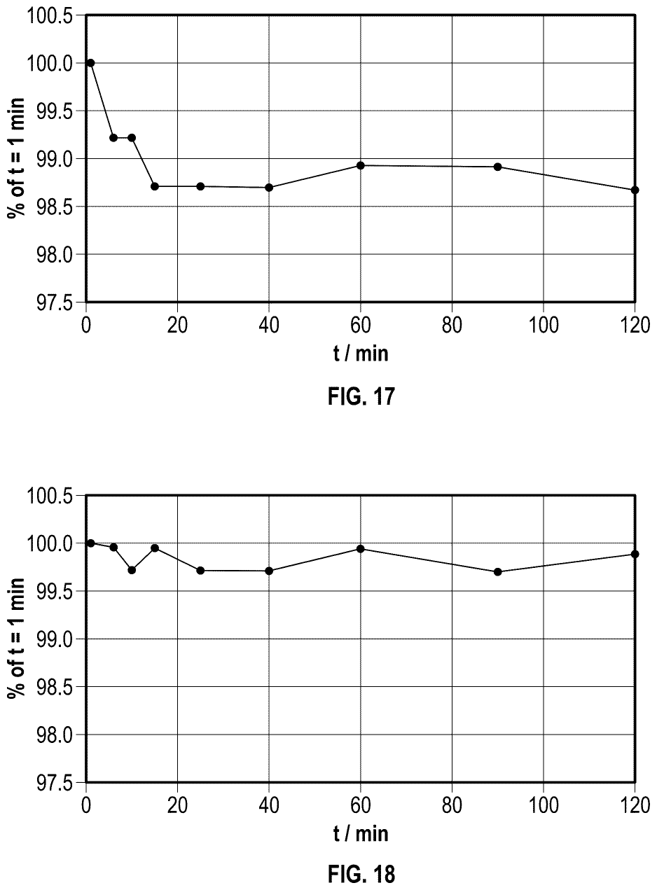

Reference is made to FIG. 17 that shows a timeseries testing for fully and partially coordinated complexes of Gd-DOTA synthesized according to the exemplary synthesis described above, treated with Ag® 50W-X8 conditioned with MegH+ resin at 1.0 molar equivalents for 2 hours, and then frozen for 77 days, then stirred at ambient temperature for 1 hour without the resin prior to the timeseries testing. The data is shown in Table 8.

It can be seen that at t=120 minutes, fully coordinated complexes are at 98.7%, and partially coordinated complexes are at 1.3% of the composition.

TABLE 8
Gd-DOTA Post Exchange Media Treatment After 77 Days Freezing,
No Secondary Exchange Media Treatment Upon Thawing

| | Time | Peak | | | Y axis | Peak | % of |
|---|---|---|---|---|---|---|---|
| | post | height | Y | | scale | height | AS15- |
| | mix/ | (274 | axis/ | Y | factor/ | (274 | 62- |
| Sample | min | nm)/cm | cm | axis/A | A/cm | nm)/A | 23(1) |
| AS15-62- | 1 | 21.15 | 19.75 | 0.40 | 0.02025 | 0.4284 | 100.0 |
| 23(1) | | | | | | | |
| AS15-62- | 6 | 21.25 | 20.00 | 0.40 | 0.02000 | 0.4250 | 99.2 |
| 23(2) | | | | | | | |
| AS15-62- | 10 | 21.25 | 20.00 | 0.40 | 0.02000 | 0.4250 | 99.2 |
| 23(3) | | | | | | | |
| AS15-62- | 15 | 21.30 | 20.15 | 0.40 | 0.01985 | 0.4228 | 98.7 |
| 23(4) | | | | | | | |
| AS15-62- | 25 | 21.30 | 20.15 | 0.40 | 0.01985 | 0.4228 | 98.7 |
| 23(5) | | | | | | | |
| AS15-62- | 40 | 21.35 | 20.20 | 0.40 | 0.01980 | 0.4228 | 98.7 |
| 23(6) | | | | | | | |
| AS15-62- | 60 | 21.40 | 20.20 | 0.40 | 0.01980 | 0.4238 | 98.9 |
| 23(7) | | | | | | | |
| AS15-62- | 90 | 21.45 | 20.25 | 0.40 | 0.01975 | 0.4237 | 98.9 |
| 23(8) | | | | | | | |
| AS15-62- | 120 | 21.45 | 20.30 | 0.40 | 0.01970 | 0.4227 | 98.7 |
| 23(9) | | | | | | | |

Reference is made to FIG. 18 that shows a timeseries testing for fully and partially coordinated complexes of Gd-DOTA synthesized according to the exemplary synthesis described above, treated with Seplife™ 50WX8-meglumineH+ resin at 1.0 molar equivalents for 5 minutes prior to the timeseries testing. The data is shown in Table 9.

It can be seen that at t=120 minutes, fully coordinated complexes are at 99.9%, and partially coordinated complexes are at 0.1% of the composition. Such a macrocyclic chelated GBCA complex, according to a mix-on-demand method according to the present disclosure, would advantageously enable administering the GBCA having only 0.1% partially coordinated complexes.

TABLE 9
Gd-DOTA with 5 Minute Exchange Media Treatment

| | Time | Peak | | | Y axis | Peak | % of |
|---|---|---|---|---|---|---|---|
| | post | height | Y | | scale | height | AS15- |
| | mix/ | (274 | axis/ | Y | factor/ | (274 | 62- |
| Sample | min | nm)/cm | cm | axis/A | A/cm | nm)/A | 13(1) |
| AS15-62- | 1 | 21.65 | 23.60 | 0.50 | 0.02119 | 0.4587 | 100.0 |
| 13(1) | | | | | | | |
| AS15-62- | 6 | 21.60 | 21.20 | 0.45 | 0.02123 | 0.4585 | 100.0 |
| 13(2) | | | | | | | |
| AS15-62- | 10 | 21.65 | 21.30 | 0.45 | 0.02113 | 0.4574 | 99.7 |
| 13(3) | | | | | | | |
| AS15-62- | 15 | 21.70 | 21.30 | 0.45 | 0.02113 | 0.4585 | 99.9 |
| 13(4) | | | | | | | |
| AS15-62- | 25 | 21.70 | 21.35 | 0.45 | 0.02108 | 0.4574 | 99.7 |
| 13(5) | | | | | | | |
| AS15-62- | 40 | 21.75 | 21.40 | 0.45 | 0.02103 | 0.4574 | 99.7 |
| 13(6) | | | | | | | |
| AS15-62- | 60 | 21.80 | 21.40 | 0.45 | 0.02103 | 0.4584 | 99.9 |
| 13(7) | | | | | | | |
| AS15-62- | 90 | 21.90 | 21.55 | 0.45 | 0.02088 | 0.4573 | 99.7 |
| 13(8) | | | | | | | |
| AS15-62- | 120 | 21.90 | 23.90 | 0.50 | 0.02092 | 0.4582 | 99.9 |
| 13(9) | | | | | | | |

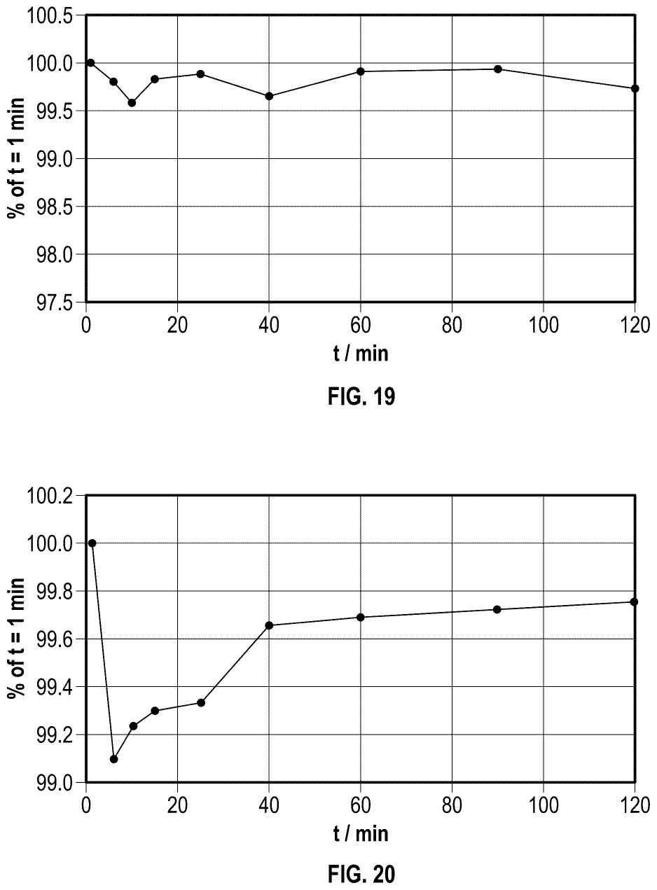

Reference is made to FIG. 19 that shows a timeseries testing for fully and partially coordinated complexes of Gd-DOTA synthesized according to the exemplary synthesis described above, treated with Seplife™ 500WX8-meglumineH+ resin at 1.0 molar equivalents for 1 minute prior to the timeseries testing. The data is shown in Table 10.

It can be seen that at t=120 minutes, fully coordinated complexes are at 99.7%, and partially coordinated complexes are at 0.3% of the composition. Such a macrocyclic chelated GBCA complex according to a mix-on-demand method according to the present disclosure would advantageously enable administering the GBCA having only 0.3% partially coordinated complexes.

TABLE 10
Gd-DOTA with 1 Minute Exchange Media Treatment

| | Peak | | | Y axis | Peak | |
|---|---|---|---|---|---|---|
| Time | height | | | scale | height | % of |
| post | (274 | Y | Y | factor/ | (274 | AS15-62- |
| mix/min | nm)/cm | axis/cm | axis/A | A/cm | nm)/cm | 18(1) |
| 1 | 19.90 | 21.00 | 0.45 | 0.02143 | 0.4264 | 100.0 |
| 6 | 20.05 | 21.20 | 0.45 | 0.02123 | 0.4256 | 99.8 |
| 10 | 20.10 | 21.30 | 0.45 | 0.02113 | 0.4246 | 99.6 |
| 15 | 20.15 | 21.30 | 0.45 | 0.02113 | 0.4257 | 99.8 |
| 25 | 20.35 | 21.50 | 0.45 | 0.02093 | 0.4259 | 99.9 |
| 40 | 20.35 | 21.55 | 0.45 | 0.02088 | 0.4249 | 99.7 |
| 60 | 20.45 | 21.60 | 0.45 | 0.02083 | 0.4260 | 99.9 |
| 90 | 20.55 | 21.70 | 0.45 | 0.02074 | 0.4262 | 99.9 |
| 120 | 20.65 | 21.85 | 0.45 | 0.02059 | 0.4253 | 99.7 |

Gadobutrol is sold under the trade name Gadovist® (Bayer AG).

A sample was exchange media treated with the C150 resin (same lot used in the time course experiments) for 25 minutes; the treated material (pH 7.22) is designated AS15-123-2. This material was used in the time course experiment AS15-121-3, the results of which are shown in FIG. 20 and Table 11 below. It can be seen that at t=120 minutes, fully coordinated complexes are about 99.75%, and partially coordinated complexes are at about 0.25% of the composition.

TABLE 11
Gadobutrol with 25 minute Exchange Media Treatment

| | | | Y axis | Peak | |
|---|---|---|---|---|---|
| | Time | Peak height | scale | height | % of |
| | post | (274 nm)/ | factor/ | (274 nm)/ | AS15- |
| Sample | mix/min | cm | A/cm | A | 121-3(1) |
| AS15-121-3(1) | 1 | 18.60 | 0.01891 | 0.3518 | 100.0 |
| AS15-121-3(2) | 6 | 18.65 | 0.01869 | 0.3486 | 99.1 |
| AS15-121-3(3) | 10 | 18.85 | 0.01852 | 0.3491 | 99.2 |
| AS15-121-3(4) | 15 | 18.95 | 0.01843 | 0.3493 | 99.3 |
| AS15-121-3(5) | 25 | 19.00 | 0.01839 | 0.3494 | 99.3 |
| AS15-121-3(6) | 40 | 19.15 | 0.01831 | 0.3506 | 99.7 |
| AS15-121-3(7) | 60 | 19.20 | 0.01826 | 0.3507 | 99.7 |
| AS15-121-3(8) | 90 | 19.25 | 0.01822 | 0.3508 | 99.7 |
| AS15-121-3(9) | 120 | 19.30 | 0.01818 | 0.3509 | 99.8 |

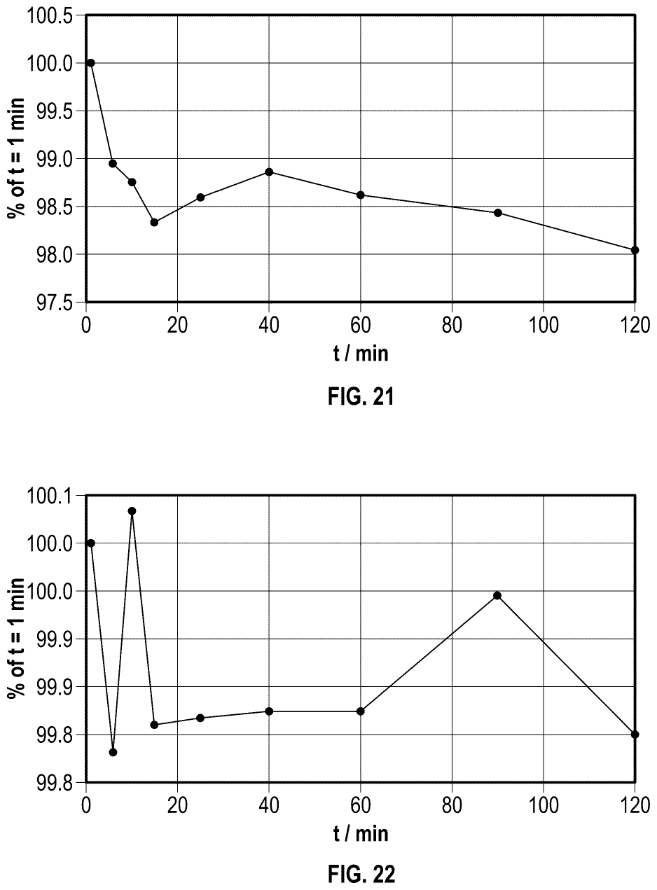

A third sample was not exchange media treated, the results of which are shown in FIG. 21 and Table 12 below. It can be seen that at t=120 minutes, fully coordinated complexes are about 98%, and partially coordinated complexes are at about 2% of the composition.

TABLE 12
Gadobutrol without Exchange Media Treatment

| | | | Y axis | Peak | |
|---|---|---|---|---|---|
| | Time | Peak height | scale | height | % of |
| | post | (274 nm)/ | factor/ | (274 nm)/ | AS15- |
| Sample | mix/min | cm | A/cm | A | 121-1(1) |
| AS15-121-1(1) | 1 | 20.50 | 0.01991 | 0.4082 | 100.0 |
| AS15-121-1(2) | 6 | 20.60 | 0.01961 | 0.4039 | 99.0 |
| AS15-121-1(3) | 10 | 20.65 | 0.01952 | 0.4031 | 98.8 |
| AS15-121-1(4) | 15 | 20.65 | 0.01944 | 0.4014 | 98.3 |
| AS15-121-1(5) | 25 | 20.75 | 0.01940 | 0.4025 | 98.6 |
| AS15-121-1(6) | 40 | 20.85 | 0.01935 | 0.4035 | 98.9 |
| AS15-121-1(7) | 60 | 20.80 | 0.01935 | 0.4026 | 98.6 |
| AS15-121-1(8) | 90 | 20.85 | 0.01927 | 0.4018 | 98.4 |
| AS15-121-1(9) | 120 | 20.90 | 0.01915 | 0.4002 | 98.0 |

Accordingly, the data demonstrates that exchange media treatment with the C150 resin can shift the composition to greater than 99% fully coordinated complexes, and that otherwise, the composition settles around 98% fully coordinated complexes.

An exchange media treatment of Dotarem® with AG50W-X12-MeglumineH+ Resin was performed. A sample of Dotarem (22.4 g) was treated with AG50W-X12-Meglumine resin (7.5 g, corresponding to two equivalents relative to Dotarem) using a 30 mL syringe like that shown in FIG. 1 as the reaction vessel. The increased resin quantity reduced the rate of resin settling compared to prior experiments. The mixture was subjected to ten manual inversions over the course of one minute to ensure adequate contact between the resin and solution. Following treatment, the liquid phase was expelled through a filter positioned at the bottom of the syringe barrel. An initial attempt with the syringe held horizontally resulted in minor resin accumulation on the filter; the liquid was drawn back into the syringe and expelled again, yielding improved flow with only slight residual resin on the needle filter. The total expulsion time was approximately 25 seconds.

Eight minutes after completion of filtration, an aliquot of the filtrate (sample AS15-78-12) was used in a subsequent time course experiment (AS15-62-29) to measure the percentage of peak height at t=1 minute as a function of time. The resulting plot is shown in FIG. 22. It can be seen that at t=120 minutes, fully coordinated complexes are about 99.8%, and partially coordinated complexes are at about 0.2% of the composition.

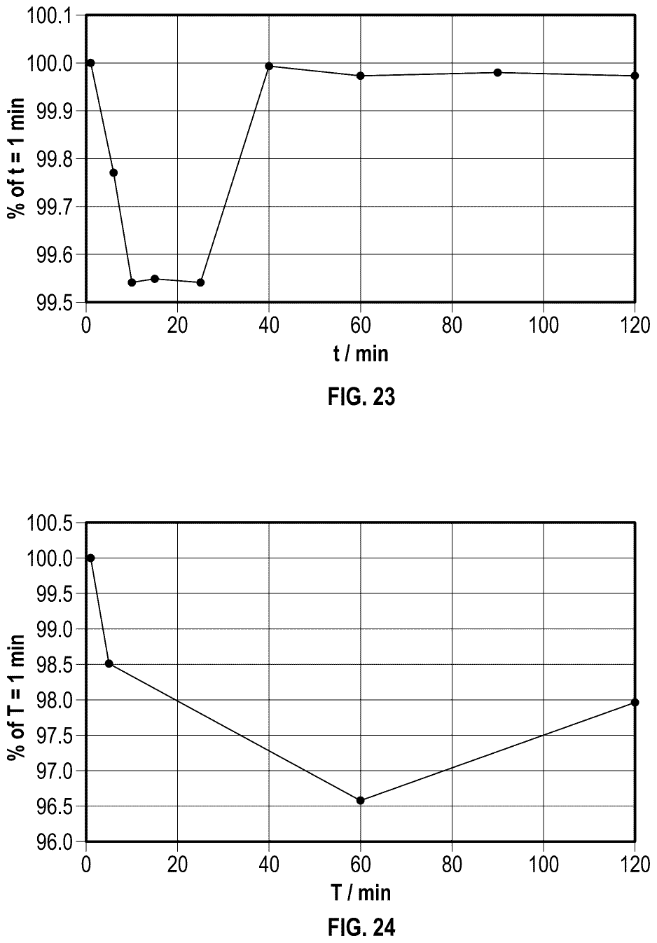

Reference is made to FIG. 23 that shows a timeseries testing for fully and partially coordinated complexes of Gd-DOTA synthesized according to the exemplary synthesis described above, treated with AG50W-X12-MegH+ resin at 1.0 molar equivalents for 20 minutes prior to the timeseries testing. The data is shown in Table 13

It can be seen that at t=120 minutes, fully coordinated complexes are at 99.95%, and partially coordinated complexes are at almost 0.05% of the composition. Such a macrocyclic chelated GBCA complex, according to a mix-on-demand method according to the present disclosure, would advantageously enable administering the GBCA having 0.05% partially coordinated complexes.

TABLE 13
Gd-DOTA with exchange media treatment

| | | | Y axis | Peak | |
|---|---|---|---|---|---|
| | Time | Peak height | scale | height | % of |
| | post | (274 nm)/ | factor/ | (274 nm)/ | AS14- |
| Sample | mix/min | cm | A/cm | A | 42-1(1) |
| AS14-42-1(1) | 1 | 21.80 | 0.02227 | 0.4855 | 100.0 |
| AS14-42-1(2) | 6 | 21.75 | 0.02227 | 0.4844 | 99.8 |
| AS14-42-1(3) | 10 | 21.70 | 0.02227 | 0.4833 | 99.5 |
| AS14-42-1(4) | 15 | 21.75 | 0.02222 | 0.4833 | 99.5 |
| AS14-42-1(5) | 25 | 21.70 | 0.02227 | 0.4833 | 99.5 |
| AS14-42-1(6) | 40 | 21.75 | 0.02232 | 0.4855 | 100.0 |
| AS14-42-1(7) | 60 | 21.60 | 0.02247 | 0.4854 | 100.0 |
| AS14-42-1(8) | 90 | 21.65 | 0.02242 | 0.4854 | 100.0 |
| AS14-42-1(9) | 120 | 21.60 | 0.02247 | 0.4854 | 100.0 |

Conditional Partial Coordination Equilibrium Constants were calculated according to:

Log k cond. (Resin-UV Comparative Protocol)=log[ML/(M)×(L)].

Unlike industry quotes of conditional stability, which are at pH 7.4 and are not in the blood, and thus in the absence of competitive chelators, these Conditional Partial Coordination Equilibrium Constant calculations are defined at pH 7.2 in the presence of a competitive chelator.

For Gd-DOTA with exchange media treatment:

log(0.5 mmol/l×0.9995)/((0.5 mmol/l×0.0005)×(0.5 mmol/l×0.0005))=6.9,

where 0.5 mmol/l is the starting concentration of gadoterate meglumine, and 0.9995 is 99.95% of gadoterate remaining by Resin-UV Comparative Protocol after exchange media treatment, and (0.5 mmol/l×0.0005) is 0.05% of the gadoterate dechelating to give this value in mmol/l of free Gd and free DOTA. In this example, there are 0.05% partially coordinated complexes and 99.95% fully coordinated complexes.

For Gd-DOTA without exchange media treatment:

log(0.5 mmol/l×0.98)/((0.5 mmol/l×0.02)×(0.5 mmol/l×0.02))=3.7.

In this example, there are 2% partially coordinated complexes and 98% fully coordinated complexes.

For Gd-DOTA with exchange media treatment, and 1% partially coordinated complex and 99% fully coordinated complexes:

log(0.5 mmol/l×0.99)/((0.5 mmol/l×0.01)×(0.5 mmol/l×0.01))=4.3

For Gd-DOTA with exchange media treatment, and 0.5% partially coordinated complex and 99.5% fully coordinated complexes:

log(0.5 mmol/l×0.995)/((0.5 mmol/l×0.005)×(0.5 mmol/l×0.005))=4.9

For Gd-DOTA with exchange media treatment, and 0.1% partially coordinated complex and 99.9% fully coordinated complexes:

log(0.5 mmol/l×0.999)/((0.5 mmol/l×0.001)×(0.5 mmol/l×0.001))=6.3

For Gadobutrol with exchange media treatment:

log(1.0 mmol/ml×0.998)/((1.0 mmol/ml×0.002)×(1.0 mmol/ml×0.002))=5.4

where 1.0 mmol/ml is the starting concentration of gadobutrol, and the concentrations ML are taken from the graphs, and the concentrations of M and L are 1.0 minus the values from the graph.

For Gadobutrol without exchange media treatment:

log(1.0 mmol/ml×0.98)/((1.0 mmol/ml×0.02)×(1.0 mmol/ml×0.02))=3.4,

where 1.0 mmol/ml is the starting concentration of gadobutrol, and the concentrations ML are taken from the graphs, and the concentrations of M and L are 1.0 minus the values from the graph.

For gadopiclinol with exchange media treatment:

log(0.5 mmol/l×0.999)/((0.5 mmol/l×0.001)×(0.5 mmol/l×0.001))=6.3

where 1.0 mmol/ml is the starting concentration of gadobutrol, and the concentrations ML are taken from the graphs, and the concentrations of M and L are 1.0 minus the values from the graph.

For gadopiclinol without exchange media treatment:

log(0.5 mmol/l×0.98)/((0.5 mmol/l×0.02)×(0.5 mmol/l×0.02))=3.7

where 1.0 mmol/ml is the starting concentration of gadobutrol, and the concentrations ML are taken from the graphs, and the concentrations of M and L are 1.0 minus the values from the graph.

This demonstrates that the exchange media treatments of the present disclosure can achieve a more than three orders of magnitude improvement (log₁₀ 1584.89≈3.2), which product can be administered to a patient or have its chelate integrity prolonged according to the disclosure. Without exchange media treatment, these improvements, including up to three orders of magnitude for Gd-DOTA and two orders of magnitude for gadobutrol, are not achieved.

Experiments were performed to determine lyophilization parameters for gadoterate meglumine after exchange media treatment.

A sample of gadoterate meglumine treated in a resin-filled syringe (two equivalents of AG50W-X12 MeglumineH+, one minute contact time) was analyzed to establish suitable lyophilization conditions. Physico-chemical characterization was conducted using electrical resistance measurements, freeze-drying microscopy, and low-temperature differential scanning calorimetry (LT-DSC).

Electrical resistance measurements identified key thermal transitions during cooling and warming cycles. Supercooling occurred to −12.2 °C before ice nucleation. A distinct phase transition was observed at −21 °C during the warming cycle, corresponding to melting and recrystallization behavior.

Freeze-drying microscopy provided direct observation of the sample during freezing and sublimation. Nucleation was detected at −10.3 °C (cell center) and −5.4 °C (cell bottom), with secondary solidification initiating at −5.6 °C. Complete solidification was achieved at −39.0 °C. Under vacuum at −60 °C, sublimation commenced at −53.8 °C, with signs of structural collapse noted below −30 °C.

LT-DSC identified key glass transition and melting events. A minor glass transition occurred at −48.2 °C (ΔCp=0.176 J/g·°C). A major glass transition occurred at an onset temperature of −32.6 °C (ΔCp=0.468 J/g·°C), indicating material flow and the onset of collapse. An endothermic melting event was observed at −27.8 °C (ΔH=166 J/g).

The data indicate that cooling below −40 °C is required for adequate solidification. Primary drying temperatures should be maintained from −37 °C and −35 °C to prevent collapse and preserve product structure. Annealing near −35 °C can elevate the glass transition temperature, potentially enabling higher primary drying temperatures in future process optimization.

An experiment was conducted to show that the contact time with the selective exchange media in mix-on-demand methods can be extended beyond one to five minutes without a detrimental effect on the final product.

Gadoterate meglumine was combined with AG50W-X12-meglumineH⁺ resin, prepared with two equivalents, and stirred at ambient temperature. Samples were collected at 1, 2, 4, 6, and 23.5 hours and analyzed by HPL. An initial decrease of approximately 2% in gadoterate concentration was observed after one hour, consistent with removal of weak cages through dechelation and/or resin uptake. From one hour through 23.5 hours, the gadoterate concentration remained stable, demonstrating that the AG50W-X12 resin effectively removes weak cages without affecting strong cages over this time period. The meglumine content and pH remained within USP specifications, and no new impurity peaks were detected in the HPLC chromatograms. The results are shown in Table 14 below.

TABLE 14
Exposure of Gadoterate Meglumine to AG50W-X12-meglumineH+ for about 24 hours

| | | | | | | | Corr. | | |
|---|---|---|---|---|---|---|---|---|---|
| | | | | | Area | Correc- | area | % of | |
| Sample | Time | | Area | Area | counts | tion | counts | compar- | Compar- |
| name | point | Compound | counts | counts | average | factor | average | ator | ator |
| AS15- | t = 0 h | gadoterate | 11019.23 | 11062.89 | 11041.06 | 1.0136 | 11191.14 | 100.0 | AS15- |
| 155-1A | | | | | | | | | 155-1A |
| | | meglumine | 2167.97 | 2340.36 | 2254.17 | 1.0136 | 2284.81 | | |
| AS15- | t = 1 h | gadoterate | 10760.19 | 10759.54 | 10759.87 | 1.0177 | 10950.33 | 97.8 | AS15- |
| 155-2A | | | | | | | | | 155-1A |
| | | meglumine | 2087.43 | 2095.52 | 2091.48 | 1.0177 | 2128.50 | | |
| AS15- | t = 2 h | gadoterate | 10668.62 | 10719.72 | 10694.17 | 1.0329 | 11045.55 | 98.7 | AS15- |
| 155-3A | | | | | | | | | 155-1A |
| | | meglumine | 2117.51 | 2092.12 | 2104.82 | 1.0329 | 2173.97 | | |
| AS15- | t = 4 h | gadoterate | 10584.37 | 10518.35 | 10551.36 | 1.0421 | 10995.37 | 98.3 | AS15- |
| 155-4A | | | | | | | | | 155-1A |
| | | meglumine | 2063.76 | 2050.21 | 2056.99 | 1.0421 | 2143.54 | | |
| AS15- | t = 6 h | gadoterate | 10955.62 | 10937.81 | 10946.72 | 1.0063 | 11016.21 | 98.4 | AS15- |
| 155-5A | | | | | | | | | 155-1A |
| | | meglumine | 2141.20 | 2032.02 | 2086.61 | 1.0063 | 2099.86 | | |
| AS15- | t = 23.5 h | gadoterate | 11186.83 | 11116.24 | 11151.54 | 0.9810 | 10940.05 | 97.8 | AS15- |
| 155-6A | | | | | | | | | 155-1A |
| | | meglumine | 1851.16 | 1761.39 | 1806.28 | 0.9810 | 1772.02 | | |

The Resin-UV Comparative Protocol is not appropriate for gadopiclinol; However, an HPLC method can be used in its place.

Gadopiclinol can be combined with approximately one molar equivalent of a C150 cation-exchange resin and agitated at ambient temperature for up to about two hours. Aliquots of the liquid phase can be withdrawn at predetermined time points, including at about 1 minute, 10 minutes, 30 minutes, 60 minutes, and 120 minutes, and separated from the resin prior to analysis.

The concentration of gadopiclinol in each aliquot can be determined by high-performance liquid chromatography (HPLC). In one exemplary analytical method, samples are analyzed using a reversed-phase C18 column with ultraviolet detection at approximately 271 nm, employing a gradient elution system comprising an aqueous phase containing trifluoroacetic acid and an organic phase containing trifluoroacetic acid in acetonitrile. Representative operating conditions include a flow rate of about 0.7 mL/min, an injection volume of about 50 μL, and a run time of about 30 minutes, although other chromatographic conditions suitable for separating gadopiclinol can also be used.

Prior to injection, samples can be diluted with water to a target gadopiclinol concentration of approximately 10 mg/mL and filtered to remove particulates. The gadopiclinol concentration at each time point can be quantified by comparison to a reference standard. Any observed decrease in gadopiclinol concentration over time is attributed to dechelation resulting from disruption of weak gadolinium chelate species upon contact with the C150 resin, thereby providing a measure of partially and fully chelated complexes.

Reference is made to FIG. 24. As with gadoterate meglumine and gadobutrol, an equilibrium exists between the strong cages and weak cages that are more prone to dechelation when competitive chelators are present.

An experiment was conducted to evaluate the stability of gadopiclenol under ion-exchange challenge conditions without exchange media treatment according to the present disclosure.

C150 cation-exchange resin (1.0 g) was combined with deionized water (5.0 g), and the mixture was allowed to stand at ambient temperature overnight to hydrate the resin. Gadopiclenol (5.0 g) was then added to the hydrated resin mixture, with completion of the addition designated as time zero (t=0). The resulting mixture was maintained at ambient temperature.

At predetermined time points following addition of gadopiclenol, aliquots of the mixture (approximately 0.5 mL each) were withdrawn and immediately filtered through a 0.45 μm syringe filter to remove resin particles. Sampling time points included approximately 1 minute, 5 minutes, 1 hour, 2 hours, 4 hours, 8 hours, and 24 hours.

For HPLC analysis, each filtered aliquot was prepared by weighing approximately 100-150 mg of sample into a vial and diluting with deionized water to achieve an overall dilution of approximately 100-fold by weight. The resulting solutions were mixed thoroughly prior to analysis.

Samples were analyzed by high-performance liquid chromatography using a reversed-phase method suitable for detection of gadopiclenol as discussed above. Under the chromatographic conditions employed, gadopiclenol appeared as a group of three closely eluting peaks that were not baseline-resolved. Because separate integration of the individual peaks was prone to integration variability, quantification was performed by integrating the combined area of the three peaks to determine the total gadopiclenol content at each time point.

A time-dependent decrease in total gadopiclenol signal was observed upon exposure to the C150 resin, indicating loss of intact gadolinium chelate species due to dechelation in the presence of a competing chelator. Over the course of the experiment, approximately 2% of the gadopiclenol was observed to break down under the challenge conditions into partially chelated complexes. The data is shown in Table 15 below.

TABLE 15
Gadopiclinol Without Exchange Media Treatment

| | | | Corr. | | |
|---|---|---|---|---|---|
| | Time- | | area | | |
| Sample | point/ | Com- | counts | % of | |
| name | min | pound | ave. | comparitor | Comparitor |
| AS16-67-1 | 1 | Sum | 20750.10 | 100.0 | AS16-67-1 |
| AS16-67-2 | 5 | Sum | 20441.17 | 98.5 | AS16-67-1 |
| AS16-67-3 | 60 | Sum | 20040.04 | 96.6 | AS16-67-1 |
| AS16-67-4 | 120 | Sum | 20326.96 | 98.0 | AS16-67-1 |

The resulting time-course profile was qualitatively similar to previously observed profiles for gadoterate meglumine without exchange media treatment subjected to comparable challenge experiments, suggesting analogous behavior with respect to the presence of partially chelated complexes.

These results support the conclusion that gadopiclenol, like gadoterate meglumine and gadobutrol, contains about 2% of partially chelated complexes that are susceptible to dechelation.

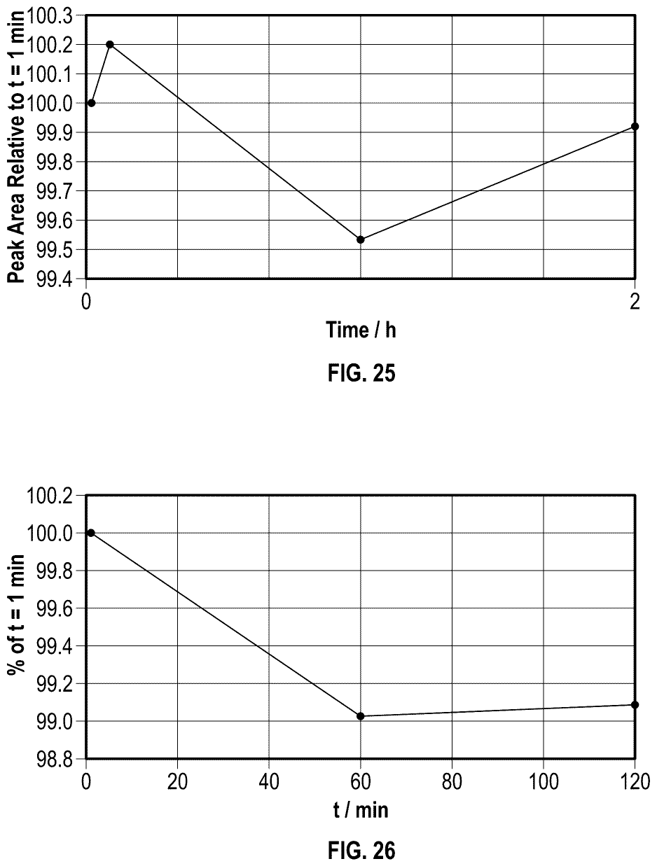

Reference is made to FIG. 25. Gadopiclinol was subjected to an exchange media treatment according to the present disclosure

Gadopiclenol was combined with AG50W-X8 cation-exchange resin in the meglumine H⁺ form and stirred at ambient temperature for approximately 2 hours and 15 minutes. Following completion of the exchange media treatment, the resin was removed by filtration using a syringe filter equipped with a 0.45 μm membrane. The resulting filtrate (designated AS16-83-0) was used within approximately five minutes to initiate a time-course experiment. At predetermined time points following filtration, aliquots of the filtrate were withdrawn for analysis. Sampling time points included approximately 1 minute, 5 minutes, 1 hour, and 2 hours, corresponding to samples designated AS16-83-1 through AS16-83-4.

Samples were prepared for high-performance liquid chromatography (HPLC) analysis by dilution with deionized water according to a standardized protocol. Gadopiclenol was quantified using an HPLC method as discussed above. The data is shown in Table 16 below.

TABLE 16
Gadopiclinol With Exchange Media Treatment using
AG50W-X8-meglumineH+ resin for 2 h 15 min
at ambient temperature

| | | | | | | Correction | Corr. | | |
|---|---|---|---|---|---|---|---|---|---|
| | | | | | Area | factor | area | % of | |
| Sample | Time- | | Area | Area | counts | (mass- | counts | compar- | Compar- |
| name | point/h | Compound | counts | counts | ave. | based) | ave. | itor | itor |
| AS16- | 0.017 | GadPic | 20243.57 | 20325.2 | 20284.39 | 0.9999 | 20282.55 | 100.00 | AS16- |
| 83-1 | | | | | | | | | 83-1 |
| AS16- | 0.083 | GadPic | 20282.99 | 20376.66 | 20329.83 | 0.9997 | 20324.67 | 100.21 | AS16- |
| 83-2 | | | | | | | | | 83-1 |
| AS16- | 1.00 | GadPic | 20109.26 | 20181.31 | 20145.29 | 1.0021 | 20187.84 | 99.53 | AS16- |
| 83-3 | | | | | | | | | 83-1 |
| AS16- | 2.00 | GadPic | 20354.26 | 20202.51 | 20278.39 | 0.9994 | 20265.85 | 99.92 | AS16- |
| 83-4 | | | | | | | | | 83-1 |

As shown in Table 16, gadopiclenol subjected to exchange media treatment exhibited a time-course profile under challenge conditions that was qualitatively similar to that previously observed for gadoterate meglumine treated under analogous conditions. In particular, the exchange media treated gadopiclenol demonstrated improved resistance to dechelation relative to untreated material, consistent with a reduction in partially chelated complexes.

Overall, the data presented in Table. 16 provides evidence that gadopiclenol behaves similarly to gadoterate meglumine when subjected to exchange media treatment and subsequent challenge, supporting the applicability of exchange media treatment to gadopiclenol.

The data further support the premise that gadopiclenol can be improved with the exchange media treatment of the present disclosure, achieving less that 1% partially chelated complexes. By using the mix-on-demand methods presented herein and considering the data obtained with gadoterate meglumine and gadobutrol, the percentage of weak structures can be reduced 10-fold.

Spray drying experiments were conducted to evaluate whether a gadoterate meglumine solution subjected to exchange media treatment could be converted into a collectable, flowable powder while maintaining a high level of single hydration state cages/strong cages (i.e., a greater than 99% fraction of fully coordinated complexes). The outlet temperature profile and time of exposure thereto was found to be a key process variable, because higher thermal exposure can reduce the fraction of the desired single-hydration or fully coordinated complexes. Further, the experiments evaluated achievable residual moisture levels in the resulting powders (i.e., “percent drying”/% moisture), and the extent to which secondary drying could further reduce moisture without unacceptable degradation, i.e., maintaining at least 99% fully chelated complexes.

Two general spray-drying gas handling configurations were used. In Runs #1-#3, the spray-drying was performed on a Buchi B-290/295 bench top lab spray dryer in a closed loop air setup. In Runs #4-#6, the spray dryer was an open loop with nitrogen drying gas setup. Prior to the spray drying, a gadoterate meglumine solution was first exposed to strong cation resin as part of exchange media treatment and then fed to a spray dryer, as in Tables 17A and 17B below.

TABLE 17A
Spray Drying of Exchange Media Treated Gd-DOTA Runs #1-#3

| | Total | | | | Inlet | Outlet | Receiver | | |
|---|---|---|---|---|---|---|---|---|---|
| | Feed | Drying | | Condenser | Temp | Temp | Temp | Moisture | % partially |
| Run | (g) | Gas | Setup | Temp (°C) | (°C) | (°C) | (°C) | (wt %) | chelated |
| 1 | 99.66 | air | closed loop | 10 | 150 | 80 | 50 | ~5 | 1% |
| 2 | 139.99 | air | closed loop | 10 | 150 | 90 | 55 | 3.56 | n.a. |
| 3 | 156.45 | air | closed loop | 10 | 170 | 100 | 60 | 2.76 | 2% |

TABLE 17B
Spray Drying of Exchange Media Treated Gd-DOTA Runs #4-#6

| | Total | | | Inlet | Outlet | Receiver | | Initial | |
|---|---|---|---|---|---|---|---|---|---|
| | Feed | Drying | | Temp | Temp | Temp | Exhaust | Moisture | % partially |
| Run | (g) | Gas | Setup | (°C) | (°C) | (°C) | RH % | (wt %) | chelated |
| 4 | 146.97 | nitrogen | open loop | 120 | 70 | 52 | 15.5 | 3.75 | 1% |
| 5 | 159.24 | nitrogen | open loop | 120 | 65 | 47 | 27.4 | 4.52 | <1% |
| 6 | 141.27 | nitrogen | open loop | 120 | 60 | 45 | 39.3 | 5.76 | <1% |

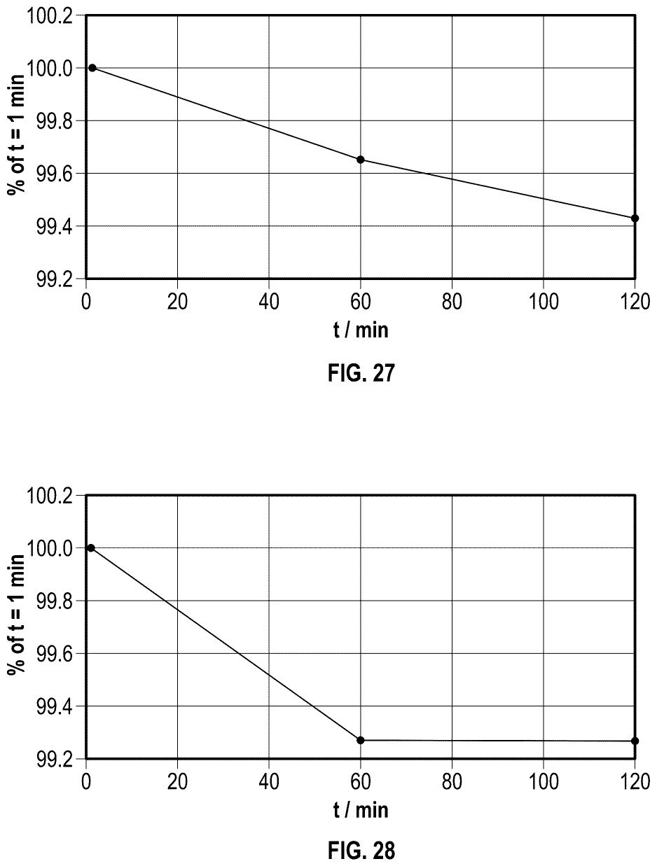

Referring to FIGS. 26, 27, and 28, spray-dried powders generated from Runs #4-#6, respectively, were evaluated using the UV timecourse protocol. These experiments show the percentage of single hydration cages in dried product after one day storage at about −50 °C. The results are also shown in Tables 18, 19, and 20 below.

TABLE 18
Scale-up batch treated w/AG50W-X8-MegH+ (1 eq.) for 2 + h;
spray-dried #4, no additional drying, 1 d freeze

| | Time | Peak | | | Y axis | Peak | |
|---|---|---|---|---|---|---|---|
| | post | height | Y | | scale | height | % of |
| | mix/ | (274 | axis/ | Y | factor/ | (274 | AS16- |
| Sample | min | nm)/cm | cm | axis/A | A/cm | nm)/A | 68-7(1) |
| AS16- | 1 | 19.70 | 21.40 | 0.50 | 0.02336 | 0.4603 | 100.0 |
| 68-7(1) | | | | | | | |
| AS16- | 60 | 20.10 | 22.05 | 0.50 | 0.02268 | 0.4558 | 99.0 |
| 68-7(7) | | | | | | | |
| AS16- | 120 | 20.25 | 22.20 | 0.50 | 0.02252 | 0.4561 | 99.1 |
| 68-7(9) | | | | | | | |

TABLE 19
Scale-up batch treated w/AG50W-X8-MegH+ (1 eq.) for 2 + h;
spray-dried #5, no additional drying, 1 d freeze

| | Time | Peak | | | Y axis | Peak | |
|---|---|---|---|---|---|---|---|
| | post | height | Y | | scale | height | % of |
| | mix/ | (274 | axis/ | Y | factor/ | (274 | AS16- |
| Sample | min | nm)/cm | cm | axis/A | A/cm | nm)/A | 68-7(1) |
| AS16- | 1 | 20.50 | 22.00 | 0.50 | 0.02273 | 0.4659 | 100.0 |
| 68-8(1) | | | | | | | |
| AS16- | 60 | 20.80 | 22.40 | 0.50 | 0.02232 | 0.4643 | 99.7 |
| 68-8(7) | | | | | | | |
| AS16- | 120 | 21.00 | 20.40 | 0.45 | 0.02206 | 0.4632 | 99.4 |
| 68-8(9) | | | | | | | |

Timecourse experiments AS16-68-7, AS16-68-8, and AS16-68-9 demonstrate that spray-dried gadoterate meglumine compositions, following one day of storage at approximately −50 °C, maintain a percentage of single-hydration-state cages above 99%. These results further show a consistent trend in which lower spray-drying temperature profiles correspond to higher retention of single-hydration-state cages. Collectively, these data establish that the disclosed gadoterate meglumine compositions can be spray dried to residual moisture levels below about 5 wt % while maintaining greater than 99% single-hydration-state cages.

TABLE 20
Scale-up batch treated w/AG50W-X8-MegH+ (1 eq.) for 2 + h;
spray-dried #6, no additional drying, 1 d freeze

| | Time | Peak | | | Y axis | Peak | |
|---|---|---|---|---|---|---|---|
| | post | height | Y | | scale | height | % of |
| | mix/ | (274 | axis/ | Y | factor/ | (274 | AS16- |
| Sample | min | nm)/cm | cm | axis/A | A/cm | nm)/A | 68-9(1) |
| AS16- | 1 | 20.55 | 20.40 | 0.45 | 0.02206 | 0.4533 | 100.0 |
| 68-9(1) | | | | | | | |
| AS16- | 60 | 20.80 | 20.80 | 0.45 | 0.02163 | 0.4500 | 99.3 |
| 68-9(7) | | | | | | | |
| AS16- | 120 | 20.95 | 20.95 | 0.45 | 0.02148 | 0.4500 | 99.3 |
| 68-9(9) | | | | | | | |

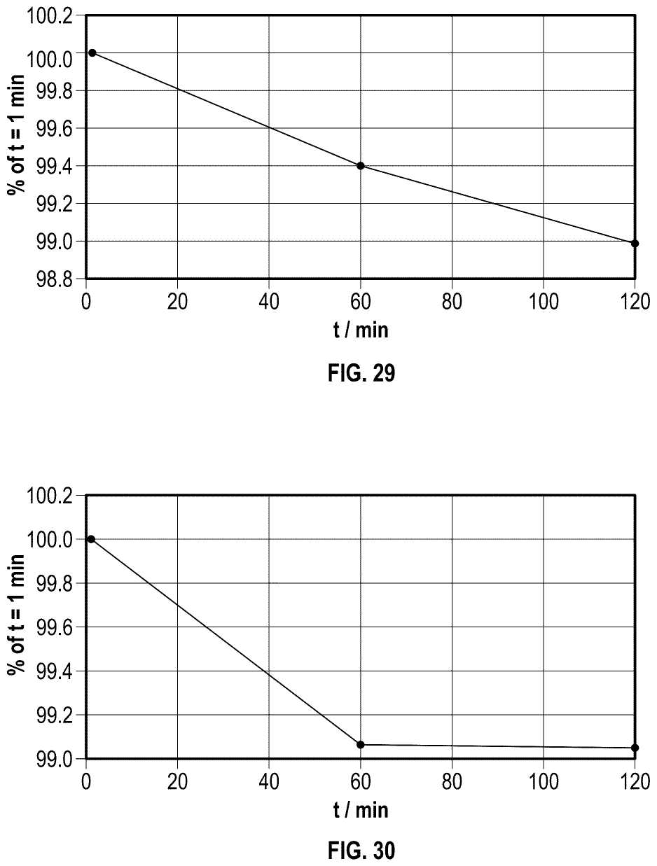

Referring to FIGS. 29 and 30, spray-dried powders generated from Runs #5 and #6, were subjected to secondary drying. The data is shown in Tables 21 and 22 below.

TABLE 21
Scale-up batch treated w/AG50W-X8-MegH+ (1 eq.) for 2 + h;
spray-dried #5, dried o/n at 30 °C and 4 h at 40 °C

| | Time | Peak | | | Y axis | Peak | % of |
|---|---|---|---|---|---|---|---|
| | post | height | Y | | scale | height | AS16- |
| | mix/ | (274 | axis/ | Y | factor/ | (274 | 68- |
| Sample | min | nm)/cm | cm | axis/A | A/cm | nm)/A | 12(1) |
| AS16- | 1 | 20.25 | 21.40 | 0.50 | 0.02336 | 0.4731 | 100.0 |
| 68-12(1) | | | | | | | |
| AS16- | 60 | 20.60 | 21.90 | 0.50 | 0.02283 | 0.4703 | 99.4 |
| 68-12(7) | | | | | | | |
| AS16- | 120 | 20.70 | 22.10 | 0.50 | 0.02262 | 0.4683 | 99.0 |
| 68-12(9) | | | | | | | |

TABLE 22
Scale-up batch treated w/AG50W-X8-MegH+ (1 eq.) for 2 + h;
spray-dried #6, dried o/n at 30 °C and 4 h at 40 °C

| | Time | Peak | | | Y axis | Peak | % of |
|---|---|---|---|---|---|---|---|
| | post | height | Y | | scale | height | AS16- |
| | mix/ | (274 | axis/ | Y | factor/ | (274 | 68- |
| Sample | min | nm)/cm | cm | axis/A | A/cm | nm)/A | 13(1) |
| AS16- | 1 | 20.80 | 22.35 | 0.50 | 0.02237 | 0.4653 | 100.0 |
| 68-13(1) | | | | | | | |
| AS16- | 60 | 21.00 | 20.50 | 0.45 | 0.02195 | 0.4610 | 99.1 |
| 68-13(7) | | | | | | | |
| AS16- | 120 | 21.15 | 20.65 | 0.45 | 0.02179 | 0.4609 | 99.0 |
| 68-13(9) | | | | | | | |

These additional timecourse experiments indicate that secondary drying results in some degradation of the single-hydration-state cage population; however, in each case, the percentage of single-hydration-state cages remains above 99%. These results indicate that secondary drying conducted for approximately 24 hours at temperatures of about 30-40 °C represents an upper bound for acceptable secondary-drying time and temperature under the tested conditions.

Based on the observed relationship between thermal exposure and cage stability, it is expected that alternative drying approaches that further reduce thermal stress, such as lyophilization or optimized vacuum spray-drying processes, would yield fully chelated complexes at comparable moisture levels, although such processes have not been experimentally demonstrated in the present examples. Likewise, improved ambient-pressure or vacuum spray-drying systems operated at commercial scale, optionally in combination with more efficient secondary-drying techniques, are expected to produce low-moisture products with minimal degradation.

Without wishing to be bound by theory, it is believed that macrocyclic gadolinium-based contrast agents subjected to exchange media treatment, or compositions in which the GBCA comprises, based on total GBCA complexes present, at least 99 mol % fully coordinated complexes in which the gadolinium ion is bound to all coordination sites of the macrocyclic chelator and not more than 1 mol % partially coordinated complexes in which the gadolinium ion is bound to fewer than all coordination sites of the macrocyclic chelator, may, when administered for magnetic resonance imaging, provide enhanced image contrast or brightness relative to compositions containing higher levels of partially coordinated complexes.

As used herein, the transitional term “comprising” is intended to be open-ended and means “including, but not limited to,” such that the recited elements can be combined with additional, unrecited elements. The transitional term “consisting of” is intended to be closed and means “including only,” such that the recited elements exclude any additional elements not expressly recited. The transitional term “consisting essentially of” is intended to be partially open and means “including the recited elements and optionally additional elements that do not materially affect the basic and novel characteristics of the claimed subject matter.” Unless the context clearly indicates otherwise, any embodiment, element, or combination of elements described herein using the term “comprising” (or grammatical variants thereof) is also expressly contemplated as an embodiment in which the transitional phrase “consisting essentially of” or “consisting of” is used in place of “comprising.” Accordingly, the present disclosure provides written description support for claims employing any of these transitional phrases, and further contemplates embodiments in which one or more additional, unrecited elements are excluded in whole or in part.

As used herein, unless explicitly stated otherwise or required by context, the terms “first”, “second”, “third”, “upper”, “lower”, and the like can be used herein to modify various elements. These modifiers do not imply a spatial, sequential, or hierarchical order to the modified elements.

As used herein, unless explicitly stated otherwise or required by context, the term “substantially” refers to the complete or nearly complete extent or degree of an action, characteristic, property, state, structure, item, or result. The exact allowable degree of deviation from absolute completeness can, in some cases, depend on the specific context. However, generally speaking, the nearness of completion will be to have the same overall result as if absolute and total completion were obtained.

As used herein, unless explicitly stated otherwise or required by context, the term “about” is used to provide flexibility to a numerical range endpoint by providing that a given value can be “a little above” or “a little below” the endpoint. Further, where a numerical range is provided, the range is intended to include any and all numbers within the numerical range, including the endpoints of the range.

As used herein, unless explicitly stated otherwise or required by context, the singular forms “a,” “an,” and “the” include plural referents, and vice versa. In particular, the terms “a” or “an,” as used in the claims and the specification, are intended to mean “at least one” or “one or more,” unless explicitly indicated otherwise. Thus, for example, reference to “an exchange resin” encompasses a single exchange resin as well as multiple exchange resins.

While the present disclosure has been described with reference to one or more exemplary embodiments, it will be understood by those skilled in the art that various changes can be made and equivalents can be substituted for elements thereof without departing from the scope of the present disclosure. In addition, many modifications can be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

1. A composition, comprising:
a macrocyclic chelated gadolinium-based contrast agent (GBCA),
wherein the GBCA comprises, based on total GBCA complexes present, at least 99% mol fully coordinated complexes in which Gd ion is bound to all coordination sites of the macrocyclic chelator, and not more than 1% mol partially coordinated complexes in which Gd ion is bound to fewer than all coordination sites of the macrocyclic chelator.

2. The composition of claim 1, wherein the GBCA is selected from the group consisting of: gadoterate, gadobutrol, gadoteridol, gadopiclenol, and gadoquatrane.

3. The composition of claim 1, wherein the composition is a dry composition suitable for reconstitution into a pharmaceutically acceptable injectable solution.

4. The composition of claim 3, wherein the GBCA is dry gadoterate meglumine having less than 5 wt % free water.

5. The composition of claim 3, further comprising:
an agent configured to, upon hydration of the GBCA, induce dechelation of Gd ions from partially coordinated complexes of the macrocyclic chelated GBCA and bind dissociated Gd ions,
wherein the GBCA and the agent are both in a dry state, and
wherein the agent remains substantially unreactive in the absence of free water and becomes effective upon hydration.

6. The composition of claim 5, wherein the agent is a trivalent-cation selective resin configured to reduce a proportion of partially coordinated complexes of the GBCA relative to a proportion of fully coordinated complexes of the GBCA.

7. A drug product comprising:
one or more containers defining at least a first chamber and a second chamber separated by a barrier;
a macrocyclic chelated gadolinium-based contrast agent (GBCA) disposed in the first chamber;
a selective exchange medium disposed in the second chamber;
wherein the barrier is configured to permit fluid communication between the first chamber and the second chamber; and
wherein the one or more containers are configured so that, upon introduction of a liquid into the one or more containers and fluid communication between the first chamber and the second chamber, the GBCA contacts the liquid and, in the presence of the liquid, contacts the selective exchange medium to provide a GBCA solution within the one or more containers.

8. The drug product of claim 7, wherein the liquid is a pharmaceutically acceptable aqueous carrier disposed in the first chamber, the second chamber, or a third chamber configured for fluid communication with the first or the second chamber.

9. The drug product of claim 7, wherein the GBCA solution, after contact with the selective exchange medium, comprises, based on total GBCA complexes present, at least 99% mol fully coordinated complexes in which Gd ion is bound to all coordination sites of the macrocyclic chelator, and not more than 1% mol partially coordinated complexes in which Gd ion is bound to fewer than all coordination sites of the macrocyclic chelator.

10. The drug product of claim 7, wherein the selective exchange medium comprises a cation exchange resin, an ion exchange membrane, a monolith, an ion exchange bead, an ion exchange particle, a porous matrix, a functionalized polymer, or a mixed-bed ion exchanger.

11. The drug product of claim 7, wherein the GBCA is selected from the group consisting of: gadoterate, gadobutrol, gadoteridol, gadopiclenol, and gadoquatrane.

12. The drug product of claim 7, wherein the GBCA is a dry composition having less than 5 wt % free water and is suitable for reconstitution into a pharmaceutically acceptable injectable solution.

13. The drug product of claim 7, wherein the selective exchange medium is configured to, upon hydration of the GBCA, induce dechelation of Gd ions from partially coordinated complexes of the macrocyclic chelated GBCA and bind dissociated Gd ions.

14. A method, the method comprising:
administering to a subject in need thereof a diagnostically effective amount of a macrocyclic gadolinium-based contrast agent (GBCA) in a pharmaceutically acceptable aqueous solution,
wherein, based on total GBCA complexes present:
at least 99% mol are fully coordinated complexes in which Gd ion is bound to all coordination sites of a macrocyclic chelator of the GBCA; and
at most 1% mol are partially coordinated complexes in which Gd ion is bound to fewer than all coordination sites of the macrocyclic chelator,
wherein the GBCA is selected from the group consisting of: gadoterate, gadobutrol, gadoteridol, gadopiclenol, and gadoquatrane.

15. A method, the method comprising:
contacting a solution comprising a macrocyclic chelated gadolinium-based contrast agent (GBCA) with an agent configured to induce dechelation of Gd ions from partially coordinated complexes of the GBCA and bind dissociated Gd ions;
separating the agent from the solution; and
administering the solution to a patient while the solution comprises a decreased proportion of partially coordinated complexes of the GBCA relative to the solution prior to the contacting.

16. The method of claim 15, wherein the agent is further configured to isolate and remove partially coordinated complexes of the GBCA in the solution.

17. The method of claim 15, wherein the agent is further configured to isolate and remove free macrocyclic chelate in the solution.

18. The method of claim 15, wherein the agent is further configured to isolate and remove partially coordinated complexes of the GBCA in the solution and free macrocyclic chelate in the solution.

19. The method of claim 15, wherein the administering is performed while at least 99% mol are fully coordinated complexes in which Gd ion is bound to all coordination sites of a macrocyclic chelator of the GBCA and at most 1% mol are partially coordinated complexes in which Gd ion is bound to fewer than all coordination sites of the macrocyclic chelator.

20. The method of claim 15, wherein the GBCA is selected from the group consisting of: gadoterate, gadobutrol, gadoteridol, gadopiclenol, and gadoquatrane.

21. A dry composition suitable for reconstitution into a pharmaceutically acceptable injectable solution, comprising:
a macrocyclic gadolinium-based contrast agent (GBCA) selected from the group consisting of: gadoterate, gadobutrol, gadoteridol, gadopiclenol, and gadoquatrane,
wherein the GBCA comprises less than 1 wt % bound water.
